SEQUENCE LISTING (9 sequences)

SEQ ID NO: 1 - DNA, 14 bp, Homo sapiens
uuccuuuguucccu

SEQ ID NO: 2 - DNA, 12 bp, Homo sapiens
gggggauauuau

SEQ ID NO: 3 - DNA, 64 bp, Homo sapiens
uuaaagguuccuuuguucccuaaguccaacuacuaaacugggggauauuaugaagggccuugag

SEQ ID NO: 4 - DNA, 75 bp, Homo sapiens
atttctattaaaggttcctttgttccctaagtccaactactaaactgggggatattatgaagggccttgagcatc

SEQ ID NO: 5 - DNA, 18 bp, Homo sapiens
gggggatattatgaaggg

SEQ ID NO: 6 - DNA, 18 bp, Homo sapiens
ttcctttgttccctaagt

SEQ ID NO: 7 - DNA, 18 bp, Homo sapiens
atgccgtaatgccgtaat

SEQ ID NO: 8 - DNA, 75 bp, Homo sapiens
atttctattaaaggttcctttgttccctaagtccaactactaaactttcctttgttccctaagtccttgagcatc

SEQ ID NO: 9 - DNA, 626 bp, Homo sapiens
acatttgcttctgacacaactgtgttcactagcaacctcaaacagacaccatggtgcatctgactcctgaggagaagtctgccgttactgccctgtggggcaaggtgaacgtggatgaagttggtggtgaggccctgggcaggctgctggtggtctacccttggacccagaggttctttgagtcctttggggatctgtccactcctgatgctgttatgggcaaccctaaggtgaaggctcatggcaagaaagtgctcggtgcctttagtgatggcctggctcacctggacaacctcaagggcacctttgccacactgagtgagctgcactgtgacaagctgcacgtggatcctgagaacttcaggctcctgggcaacgtgctggtctgtgtgctggcccatcactttggcaaagaattcaccccaccagtgcaggctgcctatcagaaagtggtggctggtgtggctaatgccctggcccacaagtatcactaagctcgctttcttgctgtccaatttctattaaaggttcctttgttccctaagtccaactactaaactgggggatattatgaagggccttgagcatctggattctgcctaataaaaaacatttattttcattgc

(19) United States
(12) Patent Application Publication
(10) Pub. No.: US 2011/0086904 A1
(43) Pub. Date: Apr. 14, 2011

(54) GENERATION OF HYPERSTABLE mRNAs

(75) Inventor: Eric J. Russell, Gladwyne, PA (US)

(73) Assignee: THE TRUSTEES OF THE UNIVERSITY OF PENNSYLVANIA, Philadelphia, PA (US)

(21) Appl. No.: 12/678,651

(22) PCT Filed: Sep. 17, 2008
(86) PCT No.: PCT/US2008/076710

§ 371 Date: Jul. 19, 2010

Related U.S. Application Data
(60) Provisional application No. 60/960,120, filed on Sep. 17, 2007.

(51) Int. Cl. A61K 31/711; C07H 21/02; C12N 5/09; A61P 7/06

(52) U.S. Cl. 514/44 R; 536/23.1; 435/367

(57) ABSTRACT

Provided herein is a method for enhancing the stability of a mRNA molecule. Specifically, the invention provides methods of increasing stability or augmenting expression of mRNA or its products by inserting a stability inducing motif at the 3′UTR of the molecule.

### FIELD OF THE INVENTION

The present invention provides a method for enhancing the stability of a mRNA molecule. Specifically, the invention provides methods of increasing stability or augmenting expression of mRNA or its products by inserting a stability inducing motif at the 3′UTR of the molecule.

### BACKGROUND OF THE INVENTION

Erythroid cells accumulate hemoglobin through a process that is critically dependent upon the high stabilities of mRNAs that encode their constituent alpha and beta-globin subunits. In vivo analyses estimate a half-life for human alpha-globin mRNA of between 24 and 60 h, while similar studies with cultured NIH 3T3 and murine erythroleukemia (MEL) cells, primary mouse hematopoietic cells, and human erythroid progenitors suggest a half-life value for human beta-globin mRNA that exceeds 16 to 20 h.

Globin mRNAs survive, and continue to translate at high levels, for as long as a week following nuclear condensation and extrusion in transcriptionally silent erythroid progenitor cells. The cis-acting determinants and trans-acting factors that participate in regulating alpha-globin mRNA stability have been identified, and the relevant molecular mechanisms have been described in detail. Mutational analyses carried out with cultured cells and with animal models clearly demonstrate the importance of the 3′ untranslated region (3′UTR) to the constitutively high stability of alpha-globin mRNA. The cis-acting pyrimidine-rich element (PRE) assembles an mRNP “alpha-complex” that comprises a member of the alpha-CP/hnRNP-E family of mRNA-binding proteins and possibly one or more additional trans-acting factors. The alpha-complex may slow alpha-globin mRNA decay by enhancing the binding of poly(A)-binding protein to the poly(A) tail. The alpha-complex may also prevent the access of an erythroid-cell-specific endoribonuclease to the alpha-PRE, mimicking mechanisms through which several nonglobin mRNAs evade endonucleolytic cleavage.

Unlike with alpha-globin mRNA, neither the cis elements nor the trans-acting factors that specify the constitutively high stability of human beta-globin mRNA have been fully described. Although several hundred mutations are known to affect beta-globin gene expression, few offer any insight into the position of a specific beta-globin mRNA stability-enhancing region or its likely mechanism. Common mutations that encode premature translation termination codons or adversely affect processing of beta-globin pre-mRNA, though accelerating its degradation, utilize mRNA-indifferent decay pathways and consequently do not illuminate the putative beta-globin mRNA-restricted mechanism(s) that defines its high baseline stability.

### SUMMARY OF THE INVENTION

In one embodiment, the invention provides a hyperstable mRNA, comprising a stability-inducing motif at the 3′UTR of the mRNA, said stability inducing motif comprising a site specific deletion and substitution of a predetermined nucleotide sequence at the 3′UTR.

The present invention provides in one embodiment, a method of increasing the stability of a mRNA molecule, comprising the step of inserting a stability inducing motif at the 3′UTR, thereby increasing the stability of a mRNA molecule.

In an additional embodiment, the present invention provides a method of increasing the amount of a mRNA molecule in a cell, comprising the step of inserting a stability inducing motif at the 3′UTR, thereby increasing the amount of a mRNA molecule in a cell.

In an additional embodiment, the present invention provides a method of producing an exogenous protein in a eukaryotic cell, comprising the step of inserting a stability inducing motif at the 3′UTR of a mRNA molecule encoding said protein, thereby producing an exogenous protein in a eukaryotic cell.

In one embodiment, the invention provides a method of treating thalassemia in a subject, comprising the step of administering to the subject a DNA construct encoding a hyperstabilized beta-globin mRNA, whereby the hyperstabilized beta-globin mRNA comprises a site specific deletion and substitution of a predetermined nucleotide sequence at the 3′UTR.

In another embodiment, the invention provides a method of treating hemoglobinopathy associated with β-globin in a subject, comprising the step of administering to the subject a DNA construct encoding a hyperstabilized beta-globin mRNA, whereby the hyperstabilized beta-globin mRNA comprises a site specific deletion and substitution of a predetermined nucleotide sequence at the 3′UTR.

In one embodiment, the invention provides a method of increasing translational efficiency of mRNA in a cell, comprising the step of inserting a stability inducing motif at the 3′UTR of said mRNA molecule, wherein said stability inducing motif comprising a site specific deletion and substitution of a predetermined nucleotide sequence at the 3′UTR.

### BRIEF DESCRIPTION OF THE DRAWINGS

The invention will be better understood from a reading of the following detailed description taken in conjunction with the drawings in which like reference designators are used to designate like elements, and in which:

FIG. 1. Unstable and stable variant beta-globin mRNAs. FIG. 1A depicts a map of conditionally expressed reporter genes encoding variant beta-globin mRNAs. pTRE-beta^{WT} contains the full-length human beta-globin gene, including native intronic, exonic, and 3′-flanking sequences (thin, thick, and intermediate gray lines, respectively), downstream of a Tet-conditional TRE promoter (dotted crosshatching). pTRE-beta^{ARE104} and pTRE-beta^{ARE130} are identical to pTRE-beta^{WT} except for a 59-bp ARE instability element (v) at either of two 3′UTR positions. FIG. 1A depicts a gel showing that a variant beta^{ARE104} mRNA is unstable in cultured cells. The intensities of the beta^{WT} bands were balanced by adjusting sample loading. C1 and C2 contain RNA from cells transfected singly with pTRE-beta^{WT} and pTRE-beta^{ARE104}, respectively. FIG. 1C depicts a graph showing ARE-mediated destabilization of beta-globin mRNA in cultured cells.

FIG. 2. two adjacent hexanucleotide mutations destabilize beta-globin mRNA in intact cultured cells. FIG. 2A depicts structures of variant beta-globin genes. The 3′UTR of the wild-type beta-globin gene (WT) is illustrated, with the TAA termination codon and AATAAA polyadenylation signal underlined. Each variant beta-globin gene (designated H100, H102, and H104, etc.) contains a site-specific AAGCTT hexanucleotide substitution encoding a HindIII recognition site. Dashes indicate identity with the WT sequence. FIG. 2B is a diagram showing the composition of DNA mixes used for mRNA stability studies in cultured cells. Mixes A to D each contain four or five variant TRE-linked beta^{H}-globin genes, including one (beta^{H100}) whose mRNA is used as a normalization control in subsequent analyses. Mix E contains a control variant beta^{H126} gene for the same purpose. FIG. 2C depicts a gel showing the relative stabilities of variant beta-globin mRNAs following transcriptional silencing of their encoding genes. HeLatTA cells transfected with DNA mixes A to E were exposed to Dox, and total RNA was recovered from aliquots following an additional 24 or 48 h of culture. RT-PCR⁺¹-amplified products were restricted with HindIII to generate differently sized DNA fragments whose quantities correspond to the levels of individual variant beta^{H} mRNAs in the original sample. Brackets emphasize the rapid interval decline in beta^{H122} mRNA (lanes 7 and 8) and beta^{H124} mRNA (lanes 9 and 10), relative to levels of other variant beta^{H} mRNAs. Lanes 1 and 2 contain ³²P-labeled size markers and the undigested PCR product from mix A, respectively. FIG. 2D depicts a graph showing the relative stabilities of variant beta^{H} mRNAs. The stabilities of individual variant beta^{H} mRNAs are plotted. Stability is defined as [(beta^{H})48/(beta^{H})24]/[(beta^{H100})48/(beta^{H100})₂₄], with the stability of beta^{H100} arbitrarily assigned unit value (subscript values represent the post-Dox intervals in hours). FIG. 2E depicts a gel showing the accelerated decay of variant beta^{H} mRNAs in intact cultured cells. The stabilities of mRNAs encoded by variant beta^{H114}, beta^{H122}, and beta^{H124}genes (top) were established singly, relative to that of internal control beta^{H100} mRNA, as described for panel C. The positions of individual HindIII-restricted RT-PCR⁺¹ product are indicated to the right. Lane 1 contains a DNA size marker. Figures F and G depict gels showing formal decay analyses of beta^{H124} and control beta^{H114} mRNAs. Mixes containing pTRE-beta^{WT} and either pTRE-beta^{H124} (F) or pTRE-^{└} H114 (G) were transfected into HeLatTA cells, and relative mRNA levels were established by RT-PCR⁺¹ at defined intervals following Dox exposure. Controls (Cont) include undigested beta^{WT} (C1), HindIII-digested beta^{WT} (C2), HindIII-digested beta^{H124} (C3), undigested beta^{H114} (C4), and HindIII-digested beta^{H114} (C5). (H) Relative stabilities of beta^{H124} and control beta^{H114} mRNAs. Band intensities were established from the autoradiographs in panels F and G by PhosphorImager densitometry. Levels of beta^{H124} and beta^{H114} mRNAs, relative to levels of coexpressed beta^{WT} mRNA and normalized to the corresponding ratio at time zero, are plotted in gray and black, respectively.

FIG. 3. Identification of a cytoplasmic factor that exhibits binding specificity for the beta^{WT} 3′UTR. FIG. 3A depicts a gel showing affinity enrichment of candidate beta-globin 3′UTR-binding factors. Agarose-immobilized ssDNAs corresponding to the 132-nt full-length beta-globin 3′UTR (beta^{WT}) or to a poly(dI·dC) negative control (NC) were incubated with K562 cytoplasmic extract, and adherent factors were resolved by SDS-PAGE. Three bands were analyzed by MALDI-TOF (asterisks). Lanes M and U contain protein size markers and unfractionated extract, respectively. FIG. 3B depicts the genetic diagramidentifying the nucleolin as a beta-globin 3′UTR-binding factor. The diagram illustrates key structural features of full-length human nucleolin, including amino-terminal acidic domains (light shading), RNA-binding domains (dark shading), and a carboxy-terminal, RGG-rich domain (crosshatched). The sizes and positions of tryptic-digest fragments, identified by MALDI-TOF analysis of affinity-enriched K562 cell extract, are indicated as black boxes below the diagram. FIG. 3C depicts a gel showing that Nucleolin (Nuc) binds liganded ssDNAs and RNAs corresponding to the beta-globin 3′UTR. K562 extract was affinity enriched using a 32-nt ligand corresponding to the H122/H124 site (32 nt) or ligands comprising the full-length (FL) beta-globin 3′UTR. Ligands comprised ssDNA, in vitro-transcribed RNA (RNA), or 2′-O-methyl RNA (Me-RNA). Poly(dI·dC) was assessed in parallel as a negative control. Lanes M and U contain protein size markers and unfractionated extract, respectively. FIG. 3D depicts a gel showing an immunological confirmation of nucleolin as a beta-globin 3′UTR-binding factor. Affinity-enriched lysate from panel A was analyzed by Western transfer analysis using nucleolin antibody MS-3. Lane U contains unfractionated extract analyzed in parallel as a migration control. FIG. 3E depicts a gel showing a sequence-specific binding of nucleolin to the beta-globin 3′UTR. Agarose immobilized ssDNAs corresponding to the beta^{WT} 3′UTR were incubated with MEL cytoplasmic extract in the presence of defined quantities of competitor poly(dI·dC). Adherent proteins were resolved on a Coomassie blue-stained SDS-polyacrylamide gel (top) and subjected to Western blot analysis using nucleolin antibody MS-3 (bottom). FIG. 3F depicts a gel showing that Nucleolin binds to the 3′UTR of beta-globin mRNA. In vitro-transcribed, ³²P-labeled RNAs corresponding to the beta^{WT} 3′UTR were incubated with total (lane T) or nucleolin-depleted (lane D) K562 extract and cross-linked with UV light, and mRNPs were resolved on a nondenaturing acrylamide gel. RNAs incubated in reconstituted lysate (lane R) and with affinity-purified nucleolin (lane C) were analyzed in parallel as controls. Bands corresponding to nucleolin-beta-3′UTR mRNPs are indicated (black spots). (Bottom) The efficiency of nucleolin depletion was assessed by Western blot analysis of reagent extracts using nucleolin antibodies (bottom). The stripped blot was rehybridized with a beta-actin antibody to control for variations in sample loading.

FIG. 4. Nucleolin is present in the cytoplasms of differentiating erythroid cells. FIG. 4A depicts a gel showing Western blot analysis performed on total (T), nuclear (N), and cytoplasmic (C) extracts prepared from MEL cells using nucleolin (Nuc) antibody. The blot was stripped and rehybridized with antibodies directed against nucleus- and cytoplasm-specific histone deacetylase-2 (HDAC-2) and beta actin, respectively. Affinity-purified nucleolin was analyzed in parallel as a positive control. FIG. 4B depicts a gel showing anucleate erythroid progenitors (reticulocytes) contain cytoplasmic nucleolin. Hemolysate prepared from FACS-sorted murine reticulocytes (Retic) was analyzed by Western transfer analysis using nucleolin antibody. Total, cytoplasmic, and nuclear extracts prepared from MEL cells were analyzed in parallel as positive controls, and recombinant alpha-CP was run as a negative control (NC). The blot was stripped and rehybridized with HDAC-2 antibody to confirm the absence of contaminating nucleoplasm in the Retic sample.

FIG. 5. Nucleolin binds to beta-globin mRNA in intact cells. FIGS. 5A and 5B depict gels showing the specificity of nucleolin-beta-globin mRNA interaction in vivo. In the experiment depicted in FIG. 5A HeLatTA cells were transfected with pTRE-beta^{WT} (beta^{WT}) or with an empty pTRE vector control (C). Total RNA recovered from cell extract (E) or nucleolin immunoprecipitate (IP) was RT-PCR amplified using beta^{WT} sequence-specific oligomers, generating a 261-bp product (lanes 2 to 5), or with GAPDH mRNA-specific oligomers, producing a 116-bp product (lanes 6 to 9). Lane 1 contains a 100-bp DNA ladder. In the experiment depicted in FIG. 5B total RNA was recovered from immunoprecipitate (lanes 3 to 5) or extract (lanes 6 and 7) prepared from cells transfected with pTRE-beta^{WT} (beta^{WT}) or with the empty pTRE vector control (C). Immunoprecipitates were prepared using nucleolin- or tumor necrosis factor-specific antibodies (Nuc or TNF, respectively). RNAs were analyzed by RNase protection using in vitro-transcribed, ³²P-labeled RNA probes. Intact and RNase-digested 32P-labeled probes were run in lanes 1 and 2, respectively. (C) Nucleolin binds beta-globin mRNA in intact human erythroid cells. Purified RNA prepared from the extract or nucleolin immunoprecipitate of density-fractionated human erythroid cells was RT-PCR amplified using human beta-globin- and GAPDH-specific oligomers. M, DNA size markers.

FIG. 6. Differential binding of nucleolin to mRNA-stabilizing and -destabilizing 3′UTR determinants. FIG. 6A depicts a gel showing beta-Globin mRNA-destabilizing that linker-scanning mutations reduce nucleolin binding in vitro. Agarose-immobilized, 59-nt ssDNAs corresponding to the proposed 3′UTR nucleolinbinding region of beta-globin mRNA were incubated in cytoplasmic extract, and adherent proteins were assessed by Western transfer analysis using nucleolin antibody. The wild-type sequence (WT) as well as sequences containing destabilizing (H124) and nondestabilizing (H120 and H126) HindIII mutations were assessed. Unfractionated extract (E) and extract adhering to unliganded agarose beads were run in the first two lanes as controls. (FIGS. 6B and C show that full-length, unstable beta^{H124} mRNA binds nucleolin poorly in vivo in intact, cultured cells. Unfractionated cell extract or nucleolin immunoprecipitate (IP) prepared from cultured cells transfected with genes encoding beta^{WT}, beta¹¹², and beta¹²⁴ mRNAs. FIG. 6B depicts a graph showing recovered RNAs that were RT-PCR amplified using primers specific to beta-globin mRNA (top) or to internal control pre-rRNA (bottom). The reaction products were resolved on an ethidium bromide-stained, nondenaturing polyacrylamide gel. Lane 1 contains a 100-bp DNA ladder. FIG. 6C depicts a gel showing recovered RNAs that were assessed by RNase protection using an in vitro-transcribed, ³²P-labeled beta-globin RNA probe.

FIG. 7. model for regulated beta-globin mRNA stability. FIG. 7A is an illustration of a secondary structure which exists within the beta-globin 3′UTR. A stable stem-loop structure within the beta-globin 3′UTR is predicted by the Zuker algorithm using default parameters. The positions of the beta-PRE and the two previously identified mRNA-destabilizing hexanucleotide mutations (H122 and H124) (gray) are indicated. FIG. 7B is an illustration of a predicted effect of the secondary structure on alpha-CP binding. The access of anto-CP to its functional beta-PRE-binding site (black) is favored by the relaxation of a native beta-globin mRNA stem-loop motif. The positioning of a binding site for nucleolin on the opposite (right) half-stem suggests a role for nucleolin in shaping the high-order 3′UTR structure. FIG. 7C depicts a graph showing RNA context-dependent binding of alpha-CP to the beta-PRE. ssDNA ligand-bound r-alpha-CP that was resolved by Coomassie blue staining after SDS-PAGE. Agarose-immobilized ligands (top), including the alpha-PRE and beta-PRE (lanes 3 and 6), the full-length beta-3′UTR (lane 5), a full-length beta-globin 3′UTR in which the beta-PRE is substituted for the alpha-PRE (lane 7), and a negative-control poly(dI·dC) (lane 4), are identified. Lanes 1 and 2 contain protein standards (M) and r-alpha-CP, respectively. FIG. 7D depicts a gel showing that alpha-CP binding to the beta-PRE is inhibited by its participation in a stable stem structure. Agarose-immobilized 2′-O-methylated RNAs corresponding to the predicted left and right half-stems (LHS and RHS, respectively) of the 3′UTR structure (32 nt each) were incubated with r-alpha-CP either singly (lanes 2 and 3) or in combination (lane 4), and adherent alpha-CP was resolved by Coomassie blue staining of SDS-PAGE gels. The LHS (black) and RHS (gray) contain the ^{└}-PRE and the H122/H124 nucleolinbinding sites, respectively. M, protein size markers. FIG. 7E depicts a gel showing that Mutations that disrupt the 3′UTR secondary structure enhance^{┘} CP binding to beta-globin mRNA. Agarose-immobilized ssDNAs were incubated with HeLa cell extract, and adherent factor was analyzed by Western blot analysis using alpha-CP antibody. The predicted structures of individual ssDNAs are schematically illustrated (top). The beta-PRE and proposed nucleolin-binding sites are represented as thick black and gray lines. Right-half-stem modifications include the deletion of a native 18-nt sequence (broken thin black line) (lane 5), the substitution of an unrelated 18-nt sequence (thin gray line) (lane 3), and the substitution of a stem-destabilizing 18-nt region containing the beta-PRE (lane 6). The unrelated stem-destabilizing sequence was analyzed as a control (lane 4). Lane 1 contains recombinant alpha-CP as a migration control (C). See Materials and Methods for details of each ssDNA sequence. FIG. 7F depicts a gel showing that Nucleolin (Nuc) enhances alpha-CP binding to the beta-globin 3′UTR in vitro. Agarose-immobilized ssDNAs corresponding to the beta-globin 3′UTR that were incubated with r-alpha-CP following no pretreatment (lane 2), heat denaturation at 95° C. for 5 min (ΔT) (lane 3), or preincubation with affinity-purified nucleolin (lane 4). Ligand-bound r-alpha-CP was analyzed by SDS-PAGE. Lane 1 contains r-alpha-CP as a migration control.

FIG. 8. Using a saturation mutagenesis approach, genes that encoded the wild-type human beta-globin mRNA were constructed, as well as additional variant β-globin genes encoding β-globin mRNAs with site-specific hexanucleotide substitutions within their 3′UTRs.

FIG. 9. The graph on the left represents the relative mRNA half lives of wild-type and two derivative beta globin constructs. Mean values from 4 or 5 separate experiments are reported. The left panel represents stylized structures of the WT construct (Top) and two different duplications of the stem-loop motif within the 3′UTR.

FIG. 10. The structures of TRE-linked beta-globin genes and their encoded mRNAs. (A) pTRE2-beta^{WT}. Left: pTRE2-β^{WT} is the full-length native human beta-globin gene with introns (thin grey lines) and exons (thick grey bars). Black vertical lines indicate translation start and stop codons. It is linked to a TRE promoter (diagonal). A 66-nt sequence corresponding to the native stem-loop structure within the 3′UTR is also shown (white). Right: The mRNA encoded by pTRE2-β^{WT} is illustrated, with the cap ( ) translation initiation and termination sites (flags) and poly(A) tail (AAA). The stem-loop structure is indicated with left and right half-stems (shaded and white, respectively). (B) pTRE2-β^{SL1} and pTRE2-β^{SL2}. Features of the gene and mRNA are described above, except that each has one additional stem-loop structure. (C) TRE2-β^{ARE}. The gene and mRNA structures are identical to those of pTRE2-β^{WT}, except for a 59-bp ARE instability element at position 15 of the 3′UTR (dark triangle).

FIG. 11. Validation of a method for assessing the stability of □-globin mRNA in situ in intact erythroid-phenotype K562 cells. (A) A real-time qRT-PCR method to measure beta-globin mRNA levels. Total cellular cDNA was prepared from K562^{tTA} cells transiently-transfected with pTRE2-beta^{WT}. Using a Cell-to-Ct kit method (Applied Biosystems), cDNA was subjected to real-time qRT-PCR amplification using Taqman probes specific for beta-globin or beta-actin endogenous control. Samples were analyzed in triplicate, using an ABI 7500 Real-Time PCR system (Applied Biosystems). The Ct values of the amplicons of both genes were within the optimal expression range. (B) Real-time qRT-PCR amplification curves of stable beta^{WT} and unstable □^{ARE} mRNAs. K562^{tTA} cells were transfected transiently with either pTRE2-beta^{WT} or pTRE2-□^{ARE} plasmids. Tetracycline was added after a 6-hour recovery period to arrest transgene transcription, and aliquots were sacrificed at 0, 1, 3, 5, 6, 8, 17 and 20 h thereafter. Representative amplification curves suggest the relative instability of the beta^{ARE} mRNA, as evidenced by the broad range of Ct values, in contrast to the narrow range of Ct values for the stable beta^{WT}. (C) The beta^{ARE} mRNA is unstable relative to beta^{WT} mRNA. The relative beta^{ARE} mRNA quantities normalized to internal control beta-actin (solid bars) decline rapidly, and are barely detectable 20 h after transcription is arrested. In contrast, the relative beta^{WT} mRNA (shaded bars) decays gradually, to >40% in 20 h. Mean values from three separate experiments are shown. (D) beta^{ARE} mRNA is one-third as stable as beta^{WT} mRNA. The bar graph indicates the calculated half-life (t½) values of beta^{ARE} mRNA relative to beta^{WT} mRNA averaged from five separate experiments. The mean t½ value of the beta^{ARE} mRNA (0.35±0.4) is three times lower than that of the beta^{WT} mRNA (1.0 relative units). This result confirms that transfected K562^{tTA} cells are clearly capable of distinguishing stable mRNAs from unstable variants encoded by conditionally-expressed genes.

### DETAILED DESCRIPTION OF THE INVENTION

In one embodiment, provided herein is a method for enhancing the stability of a mRNA molecule. In another embodiment, provided herein are methods of increasing stability or augmenting expression of mRNA or its products by inserting a stability inducing motif at the 3′ UTR of the molecule.

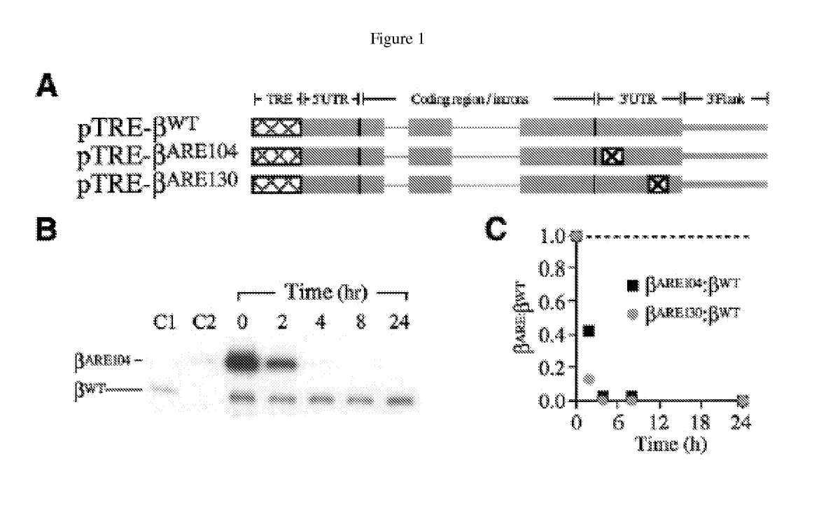

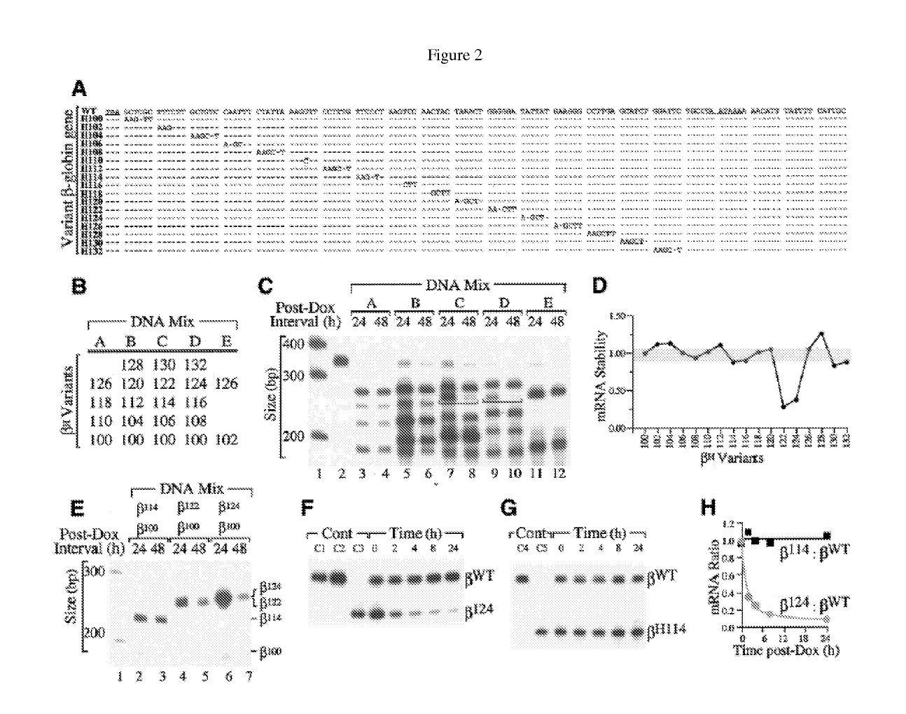

In one embodiment, the stability of human beta-globin mRNA requires cis determinants and trans-acting factors. In another embodiment, provided herein is an important method for assessing the stability of an mRNA in vivo in intact cultured cells without affecting the expression or function of other cellular mRNAs (FIG. 1). Using this approach, a defined 3′UTR region was identified, that is critical to normal beta-globin mRNA stability (FIG. 2), thus linking this important functional characteristic to a discrete, previously unrecognized structural determinant. In another embodiment other cis elements participate in this process. In one embodiment, the critical nature of the H122-H124 region; GGGGGATATTAT (SEQ ID No. 10) to beta-globin mRNA stability is clear.

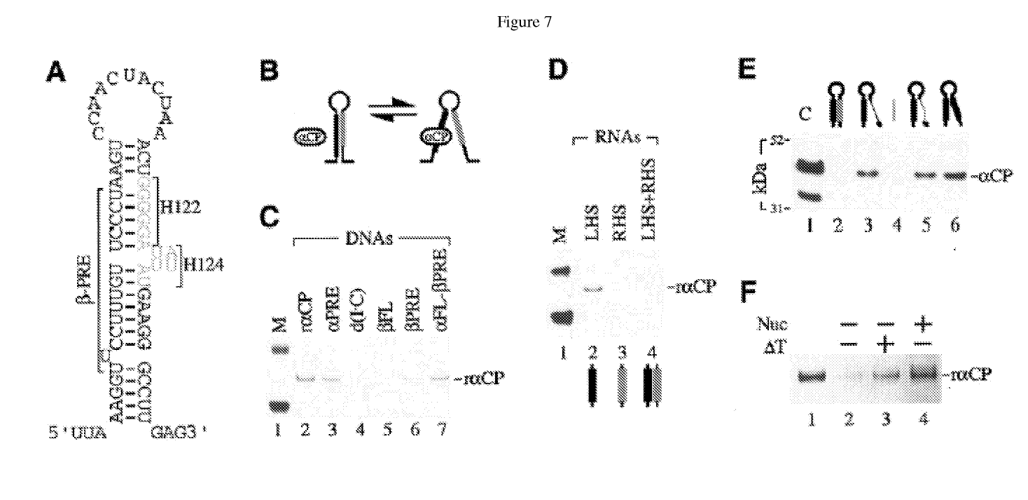

In one embodiment, provided herein is a hyperstable mRNA, comprising a stability-inducing motif at the 3′UTR of the mRNA, said stability inducing motif comprising a site specific deletion and substitution of a predetermined nucleotide sequence at the 3′UTR. In another embodiment, the deletion and substitution is applied to the 3′ UTR of the mRNA sequence in order to insert a cis-acting pyrimidine-rich element (PRE), or a nucleolin binding element in another embodiment, or both in yet another embodiment. In one embodiment the stability inducing motif is capable of forming a stem-loop construct, wherein the PRE is inserted at the left stem portion and the nucleolin binding element is inserted at the right hand side of the stem forming sequence of the stem-loop construct (see e.g. FIG. 7A).

In one embodiment, provided herein is a method of treating thalassemia in a subject, comprising the step of administering to the subject a DNA construct encoding a hyperstabilized beta-globin mRNA, whereby the hyperstabilized beta-globin mRNA comprises a site specific deletion and substitution of a predetermined nucleotide sequence at the 3′UTR.

In another embodiment, provided herein is a method of treating hemoglobinopathy associated with β-globin in a subject, comprising the step of administering to the subject a DNA construct encoding a hyperstabilized beta-globin mRNA, whereby the hyperstabilized beta-globin mRNA comprises a site specific deletion and substitution of a predetermined nucleotide sequence at the 3′UTR.

In one embodiment, provided herein is a method of quantifying the stability of mRNA variants in a cell, comprising the step of transfecting the cell with a tetracycline-regulated transactivator (tTA) fusion protein; linking a gene of interest in the cell to a recombinant hybrid tetracycline response element (TRE); contacting the cell with an effective amount of tetracycline or doxycycline (Dox); and analyzing the rate of decline in the levels of the mRNA of the recombinant hybrid tetracycline response element (TRE)-linked gene, wherein the higher the rate of decline, the less stable is the mRNA.

In another embodiment, provided herein is a method of increasing translational efficiency of mRNA in a cell, comprising the step of inserting a stability inducing motif at the 3′UTR of said mRNA molecule, wherein said stability inducing motif comprising a site specific deletion and substitution of a predetermined nucleotide sequence at the 3′UTR.

In one embodiment, provided herein is a method of increasing the stability of a mRNA molecule, comprising the step of inserting a stability inducing motif at the 3′UTR, thereby increasing the stability of a mRNA molecule. In another embodiment, increasing the stability of a mRNA molecule comprises increasing t_{1/2} of a mRNA molecule. In another embodiment, increasing the stability of a mRNA molecule comprises increasing the time period wherein the mRNA molecule is functional.

In another embodiment, inserting a stability inducing motif at the 3′UTR of a mRNA molecule results in a stability increase of a mRNA molecule by at least 1.5 folds. In another embodiment, inserting a stability inducing motif at the 3′UTR of a mRNA molecule results in a stability increase of a mRNA molecule by at least 2 folds. In another embodiment, inserting a stability inducing motif at the 3′UTR of a mRNA molecule results in a stability increase of a mRNA molecule by at least 3 folds. In another embodiment, inserting a stability inducing motif at the 3′UTR of a mRNA molecule results in a stability increase of a mRNA molecule by at least 4 folds. In another embodiment, inserting a stability inducing motif at the 3′UTR of a mRNA molecule results in a stability increase of a mRNA molecule by at least 5 folds. In another embodiment, inserting a stability inducing motif at the 3′UTR stem-loop structure of a mRNA molecule results in a stability increase of a mRNA molecule by at least 10 folds. In another embodiment, inserting a stability inducing motif at the 3′UTR stem-loop structure of a mRNA molecule results in a stability increase of a mRNA molecule by at least 15 folds.

In another embodiment, inserting a stability inducing motif at the 3′UTR stem-loop structure of a mRNA molecule results in a stability increase of a mRNA molecule by at least 20 folds. In another embodiment, inserting a stability inducing motif at the 3′UTR stem-loop structure of a mRNA molecule results in a stability increase of a mRNA molecule by at least 30 folds. In another embodiment, inserting a stability inducing motif at the 3′UTR stem-loop structure of a mRNA molecule results in a stability increase of a mRNA molecule by at least 40 folds. In another embodiment, inserting a stability inducing motif at the 3′UTR stem-loop structure of a mRNA molecule results in a stability increase of a mRNA molecule by at least 50 folds. In another embodiment, inserting a stability inducing motif at the 3′UTR stem-loop structure of a mRNA molecule results in a stability increase of a mRNA molecule by at least 60 folds. In another embodiment, inserting a stability inducing motif at the 3′UTR stem-loop structure of a mRNA molecule results in a stability increase of a mRNA molecule by at least 80 folds. In another embodiment, inserting a stability inducing motif at the 3′UTR stem-loop structure of a mRNA molecule results in a stability increase of a mRNA molecule by at least 100 folds.

In another embodiment, the mRNA molecule is encoded by a desired gene. In another embodiment, the desired gene is taken out of the DNA of the donor cell. In another embodiment, the desired gene is taken out of the DNA of a plasmid comprising the desired gene. In another embodiment, the desired gene is obtained from any genomic source known to one of skill in the art. In another embodiment, the methods of obtaining, isolating, and/or inserting the desired gene to an appropriate vector are known to one of skill in the art.

In another embodiment, the DNA molecule encoding the desired gene comprises a stability inducing motif. In another embodiment, the DNA molecule encoding the desired gene is engineered to comprise a stability inducing motif. In another embodiment, the DNA molecule encoding the desired gene is engineered to comprise a stability inducing motif at the 3′UTR. In another embodiment, the DNA molecule encoding the desired gene comprising a stability inducing motif, further comprises a promoter. In another embodiment, the promoter is a constitutively active promoter. In another embodiment, the promoter is an inducible promoter. In another embodiment, the promoter is a constitutively active promoter. In another embodiment, the promoter is a CMV promoter. In another embodiment, the DNA molecule comprises a distal promoter and a proximal promoter.

In another embodiment, the stability inducing motif comprises the nucleic acid sequence 5′-UUCCUUUGUUCCCU-'3 set forth in SEQ ID NO: 1. In another embodiment, the stability inducing motif comprises a sequence having at least 60% identity with SEQ ID NO: 1. In another embodiment, the stability inducing motif comprises a sequence having at least 70% identity with SEQ ID NO: 1. In another embodiment, the stability inducing motif comprises a sequence having at least 80% identity with SEQ ID NO: 1. In another embodiment, the stability inducing motif comprises a sequence having at least 90% identity with SEQ ID NO: 1. In another embodiment, the stability inducing motif comprises a sequence having at least 95% identity with SEQ ID NO: 1. In another embodiment, the stability inducing motif comprises a sequence having at least 98% identity with SEQ ID NO: 1.

In another embodiment, the stability inducing motif comprises the following nucleic acid sequence 5′-GGGGGAUAUUAU-'3 (SEQ ID NO: 2). In another embodiment, the stability inducing motif comprises a sequence having at least 60% identity with SEQ ID NO: 2. In another embodiment, the stability inducing motif comprises a sequence having at least 70% identity with SEQ ID NO: 2. In another embodiment, the stability inducing motif comprises a sequence having at least 80% identity with SEQ ID NO: 2. In another embodiment, the stability inducing motif comprises a sequence having at least 90% identity with SEQ ID NO: 2 In another embodiment, the stability inducing motif comprises a sequence having at least 95% identity with SEQ ID NO: 2. In another embodiment, the stability inducing motif comprises a sequence having at least 98% identity with SEQ ID NO: 2.

In another embodiment, the stability inducing motif comprises the following nucleic acid sequence 5′-UUAAAGGUUCCUUUGUUCCCUAAGUCCAACUACUAAACUGGGGGAUAUUAUGAAG GGCCUUGAG-'3 (SEQ ID NO: 3). In another embodiment, the stability inducing motif comprises a sequence having at least 60% identity with SEQ ID NO: 3 In another embodiment, the stability inducing motif comprises a sequence having at least 70% identity with SEQ ID NO: 3. In another embodiment, the stability inducing motif comprises a sequence having at least 80% identity with SEQ ID NO: 3. In another embodiment, the stability inducing motif comprises a sequence having at least 90% identity with SEQ ID NO: 3 In another embodiment, the stability inducing motif comprises a sequence having at least 95% identity with SEQ ID NO: 3. In another embodiment, the stability inducing motif comprises a sequence having at least 98% identity with SEQ ID NO: 3.

In another embodiment, the stability inducing motif comprises SEQ ID NO: 1 and SEQ ID NO:2 or sequences having a degree of identity as provided hereinabove.

In one embodiment a defined 3′UTR region that is critical to normal beta-globin mRNA stability (FIG. 2), thus linking this important functional characteristic to a discrete, previously unrecognized structural determinant. In another embodiment, other cis elements participate in this process, since the critical nature of the H122-H124 region to beta-globin mRNA stability is clear.

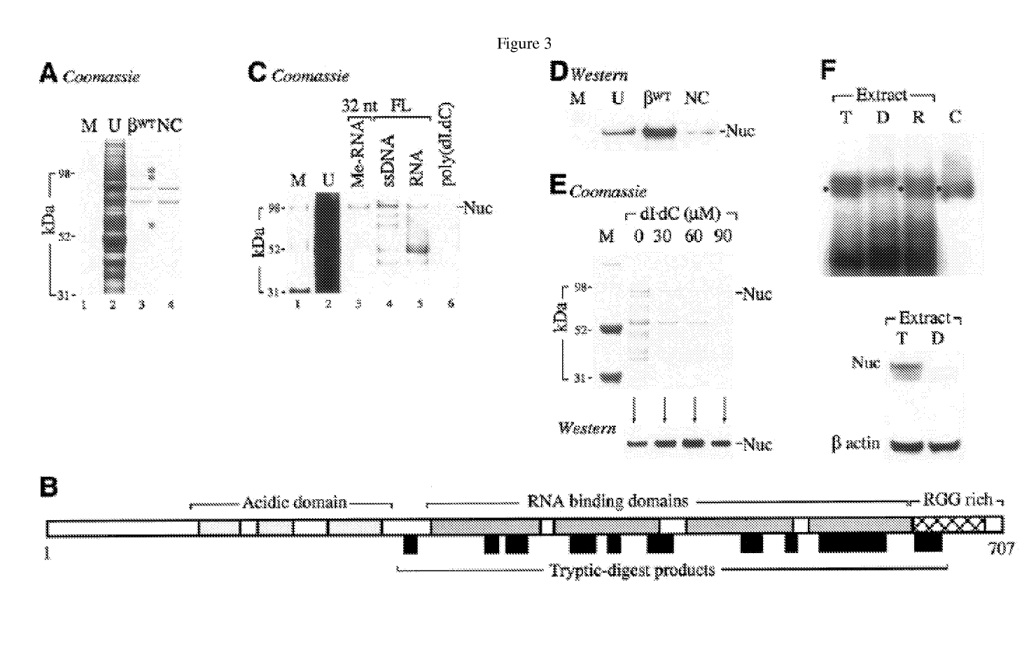

In one embodiment, nucleolin plays a central role in stabilizing beta-globin mRNA in vivo. Nucleolin displays a relative specificity for ssDNAs corresponding to the beta-globin 3′UTR in vitro (FIG. 3) and in another embodiment, interacts with full-length beta-globin mRNA both in intact cultured cells and in primary human erythroid progenitors (FIG. 5).

Among three candidate 3′ UTR-binding factors, nucleolin plays in one embodiment, a central role in stabilizing beta-globin mRNA in vivo. Nucleolin displays a relative specificity for ssDNAs corresponding to the beta-globin 3′UTR in vitro (FIG. 3) and interacts in another embodiment with full-length beta-globin mRNA both in intact cultured cells and in primary human erythroid progenitors (FIG. 5). In another embodiment, binding is ablated in vivo by mRNA-destabilizing mutations but preserved in beta-globin mRNAs carrying control nondestabilizing mutations, firmly linking nucleolin binding to its proposed mRNA-stabilizing function (FIG. 6).

The structural analyses are consistent with this possibility; in one embodiment, In one embodiment of the stability inducing motif, nucleolin binds to the right half-stem of a stable 3′UTR stem-loop structure, directly opposite to the beta-PRE (FIG. 7A). Nucleolin binding is required in another embodiment, to relax a stem-loop structure that is predicted to interfere with alpha-CP binding (FIG. 7B). In one embodiment enhanced CP binding to 3′UTRs is shown, in which the stem-loop structure is disrupted (FIG. 7C to E). In another embodiment the specific role of nucleolin in this process is by the fact that alpha-CP binding to the beta-globin 3′UTR is enhanced either by heat denaturation or by preincubation with immunopurified nucleolin (FIG. 7F).

In one embodiment, nucleolin facilitates functional interaction of other, known globin mRNA-stabilizing factors, such as αCP. In one embodiment, nucleolin binds to the right half-stem of a stable 3′UTR stem-loop structure, directly opposite to the β-PRE (FIG. 7A). In another embodiment, nucleolin binding is required to relax a stem-loop structure that is predicted to interfere with αCP binding (FIG. 7B). In vitro studies show enhanced αCP binding to 3′UTRs in which the stem-loop structure is disrupted (FIG. 7C to E), consistent with the proposed mechanism. A specific role for nucleolin in this process is shown in one embodiment by the demonstration that αCP binding to the beta-globin 3′UTR can be enhanced either by heat denaturation or by preincubation with immunopurified nucleolin (FIG. 7F).

In one embodiment, the role nucleolin plays in stabilizing beta-globin mRNA is consistent with its participation in a wide range of molecular processes. In the nucleus, nucleolin is associated with ribosome biogenesis, chromatin remodeling, immunoglobulin isotype switching, telomere formatting, and posttranscriptional processing of nascent mRNAs. In the cytoplasm, nucleolin binds to the 5′ and 3′ UTRs of specific mRNAs, enhancing both their stabilities and their translational efficiencies.

In another embodiment the proposed model whereby a stem loop structure in the 3′UTR comprising a nucleolin binding sequence at the right stem, to be particularly attractive because it accommodates both the data provided herein, and evidence from previous studies favoring a critical role for alpha-CP in stabilizing the beta-globin mRNA.

Functional diversity reflects in certain embodiments, both the complexity of the nucleolin core structure and the heterogeneity of isoforms that it can assume. The core structure, which comprises acidic and glycine rich domains as well as four RNA-binding domains (RBDs), is extensively modified by targeted proteolysis, phosphorylation, ADP ribosylation, and methylation, resulting in combinatorial structural complexity that may form the basis for its observed functional heterogeneity.

The four centrally positioned RBDs of nucleolin mediate its interaction with RNA both in the nucleus and in the cytoplasm. These domains, which are structurally similar to RBDs in protein factors that regulate the stabilities and translational efficiencies of other mRNAs, subserve in certain embodiments, a parallel spectrum of functions in nucleolin. In one embodiment, nucleolin stabilizes mRNAs encoding amyloid precursor protein, renin, CD154, and Bcl-2 by binding to structurally distinct cis elements within their 3′UTRs. In another embodiment, the heterogeneity in its posttranslational modification accounts for nucleolin's equally heterogeneous mRNA-binding specificities. The nucleolin-binding sites of interleukin 2 and amyloid precursor protein mRNAs, which share a common 5′ CUCUCUUUA 3′ (SEQ ID No. 11) target sequence, differ from the A/U-rich nucleolin-binding site in the 3′UTR of Bcl-2 mRNA and from the 5′ UCCCGA 3′ motif mediating its binding to rRNA. Nucleolinmay also bind to motifs corresponding to splice acceptor sequences (5′ UUAGG 3′) and to G-quartet and other related nonlinear, thermodynamically favorable nucleic acid structures that are not predicted by common mRNA-folding algorithms. The beta-globin mRNA nucleolin-binding determinant described (FIG. 2), is dissimilar to each of these linear elements, possibly reflecting interaction with a subset of nucleolin structural isoforms that carry specific phosphoryl, ADP-ribosyl, or methyl modifications.

In one embodiment, the stem-loop nucleotide constructs described herein are interchangeable with the hairpin structure described. In one embodiment, provided herein are methods for increasing the stability of mRNA molecules, comprising the step of inserting a hairpin structure comprising the nucleotide sequence set forth in SEQ. ID Nos. 1-3, or their combination at the 3′UTR of the mRNA molecule. In another embodiment, the hairpin structure inserted is a duplicate of a wild type hairpin structure disposed at the 3′UTR of the mRNA, wherein the additionally inserted hairpin structure is added at the 3′ side or the 5′ side of the WT hairpin structure. In one embodiment, the stability inducing motif inserted in the hyperstable mRNA molecules described herein, is a stem-loop construct comprising SEQ ID NO. 1, or SEQ ID No. 2 in another embodiment, or SEQ ID No. 3 in another embodiment or their combination, is inserted at the ′3UTR of the mRNA molecule, at a predetermined location on the 5′ side of the wild-type existing stability inducing motif.

The wide variety of molecular processes that require nucleolin indicate in one embodiment that it serves as a molecular scaffold or a substrate-remodeling factor in another embodiment, acting in concert with other proteins that provide the required functional specificity. In one embodiment a specific nucleolin-beta-globin mRNP has to assemble before alpha-CP can bind, and subsequently stabilize, the full-length beta-globin mRNA. This hypothesis explains in one embodiment the difficulties encountered in attempting to demonstrate bimolecular interactions.

The constitutive stability of β-globin mRNA in definitive erythroid cells is regulated in one embodiment, by two distinct elements within its 3′-untranslated region (3′UTR). In another embodiment, the baseline stability is enhanced by gain-of-function mutations comprising substitution, deletion, or duplication of one or both regions. Such ‘hyperstable’ β-globin mRNAs accumulate in another embodiment to high levels, increasing the expression of β globin from therapeutic transgenes that have previously been transcriptionally optimized. In one embodiment, these transgenes are important for the treatment of sickle cell disease and β-thalassemia.

In one embodiment, provided herein is a rapid and highly reproducible method for testing the stabilities of β-globin mRNAs carrying site-specific mutations within their 3′UTRs was developed. In one embodiment, the method comprises (a) a K562 cell culture system in which transcription of transiently transfected test genes can be rapidly silenced (permitting mRNA stabilities to be determined using a transcriptional chase approach), and (b) real-time RT-PCR for sensitive and accurate quantitation of individual mRNAs. Derivative human β-globin genes, containing site-specific mutations in their 3′UTRs, are transiently transfected in another embodiment into K562 cells expressing the tetracycline-dependent transcriptional transactivator (tTA) protein. Following a 24-hour recovery period, cells were exposed to tetracycline to arrest transgene transcription, and cell aliquots sacrificed at defined intervals. Total RNA, prepared using a high-throughput 96-well RNA isolation method, was subsequently subjected to real-time RT-PCR analyses using amplification/reporter Taqman probe sets for β-globin and β-actin mRNA. β-globin mRNA levels were established by ΔΔCt analysis using β-actin as endogenous reference; half-life values were derived by standard analyses of mRNA decay curves.

Validation experiments are conducted in one embodiment, using the wild-type β-globin gene and the unstable derivative β^{ARE} gene described herein. In these studies the wild-type β-globin mRNA exhibited a half-life value nearly three times greater than the unstable control mRNA (5.6±0.1 h vs 2.2±0.1 h, respectively), confirming the utility of the new method. The stabilities of derivative β-globin mRNAs carrying site-specific mutations in their 3′UTRs are assessed in one embodiment, using the methods provided herein. In one embodiment, the stability of β-globin mRNAs carrying two different duplications of a defined 3′UTR stem-loop motif previously identified as a determinant of mRNA stability is significantly increased (7.1±0.6, and 9.4±0.6 h, respectively).

Accordingly and in one embodiment, provided herein is a method of quantifying the stability of mRNA variants in a cell, comprising the step of transfecting the cell with a tetracycline-regulated transactivator (tTA) fusion protein; linking a gene of interest in the cell to a recombinant hybrid tetracycline response element (TRE); contacting the cell with an effective amount of tetracycline or doxycycline (Dox); and analyzing the rate of decline in the levels of the mRNA of the recombinant hybrid tetracycline response element (TRE)-linked gene, wherein the higher the rate of decline, the less stable is the mRNA.

In another embodiment, provided herein is a method of increasing the stability, or augmenting ex-vivo expression of a gene of interest, whose mRNA comprises a stem-loop structure associated with the stability of the mRNA molecule, comprising the step of at least duplicating the stem-loop construct at the 3′ UTR of the mRNA molecule, thereby increasing the stability of the mRNA molecule, reducing its degradation and increasing its expression.

In one embodiment, the hairpin constructs described in the methods provided herein, are used to increase the stability of mRNA molecules which do not contain a WT hairpin structure.

In another embodiment, the desired gene undergoes artificial recombination in a test tube. In another embodiment, the desired gene is inserted into a virus. In another embodiment, the desired gene is inserted into a bacterial plasmid. In another embodiment, the desired gene is inserted into any other vector system known to one of skill in the art. In another embodiment, subsequent incorporation of chimeric molecules into a host cell in which they are capable of continued propagation is performed.

In another embodiment, the methods provided herein involve joining of the DNA encoding the desired gene with a DNA vector (also known as a vehicle or a replicon) capable of autonomous replication in a living cell after foreign DNA has been inserted into it. In another embodiment, the methods provided herein involve transfer, via transformation or transfection, of the recombinant molecule into a suitable host.

In another embodiment, a suitable host is a solitary cell. In another embodiment, a suitable host is a multi-cellular organism.

In another embodiment, DNA encoding the desired gene is excised and isolated using DNA restriction enzymes such as restriction endonucleases that make possible the cleavage of high-molecular-weight DNA. In another embodiment, the restriction enzymes are type II restriction endonucleases or DNAases that recognize specific short nucleotide sequences (usually 4 to 6 base pairs in length), and then cleave both strands of the DNA duplex, generating discrete DNA fragments of defined length and sequence which comprise a DNA fragment encoding the desired gene.

In another embodiment, the DNA fragment encoding the desired gene can be easily resolved as bands of distinct molecular weights by agarose gel electrophoresis. In another embodiment, the DNA fragment encoding the desired gene is identified by Southern blotting. In another embodiment, the DNA fragment encoding the desired gene is purified prior to cloning thus, reducing the number of recombinants that must later be screened.

In another embodiment, the method that has been used to generate small DNA fragments is mechanical shearing, intense sonification of high-molecular-weight DNA with ultrasound, or high-speed stirring in a blender, can both be used to produce DNA fragments of a certain size range. In another embodiment, shearing results in random breakage of DNA, producing termini consisting of short, single-stranded regions. Other sources include DNA complementary to poly(A) RNA, or cDNA, which is synthesized in the test tube, and short oligonucleotides that are synthesized chemically.

In another embodiment, the different components/DNA fragments (stability inducing motif sequences, promoter sequences, etc.) comprised within the DNA molecule encoding the desired gene are joined. In another embodiment, the different components/DNA fragments and the vector which carry them are joined by the enzyme DNA ligase. In another embodiment, the intact engineered vector comprises a recombinant DNA duplex molecule. In another embodiment, the DNA duplex molecule is used for transformation and the subsequent selection of cells containing the recombinant molecule.

In another embodiment, the different components/DNA fragments (stability inducing motif sequences, promoter sequences, etc.) comprised within the DNA molecule encoding the desired gene are joined by the addition of homopolymer extensions to different DNA fragments followed by an annealing of complementary homopolymer sequences.

In another embodiment, the enzyme T4 DNA ligase carries out the intermolecular joining of DNA substrates at completely base-paired ends. In another embodiment, the desired DNA sequences comprising the desired gene, stability inducing motifs, and a promoter once attached to a DNA vector, are transferred to a suitable host. In another embodiment, transformation comprises the introduction of foreign DNA into a recipient cell. In another embodiment, the desired DNA sequences comprising the desired gene, stability inducing motifs, and a promoter once attached to a DNA vector, are transfected by a virus.

In another embodiment, the desired DNA sequences comprising the desired gene, stability inducing motifs, and a promoter are transformed separately into a host cell. In another embodiment, a vector comprising the joined desired DNA sequences comprising the desired gene, stability inducing motifs, and a promoter is transformed as a single cassette into a host cell.

In another embodiment, transformation results in the stable integration of the joined desired DNA sequences into a chromosome. In another embodiment, transfection results in the stable integration of the joined desired DNA sequences into a chromosome. In another embodiment, transformation results in the stable integration of a desired DNA sequence into a chromosome. In another embodiment, transformation results in the maintenance of the DNA as a self-replicating entity. In another embodiment, transfection results in the maintenance of the DNA as a self-replicating entity.

In another embodiment, the methods as described herein make use of Escherichia coli as the host for cloning. In another embodiment, the methods comprise transformation of E. coli. In another embodiment, the methods comprise E. coli treated with calcium chloride to take up DNA from bacteriophage lambda as well as plasmid DNA.

In another embodiment, the methods as described herein make use of Bacillus species. In another embodiment, the methods comprise transformation of Bacillus species comprising polyethylene glycol-induced DNA uptake. In another embodiment, the methods as described herein make use of Actinomycetes that can be similarly transformed. In another embodiment, transformation is achieved by first entrapping the DNA with liposomes followed by their fusion with the host cell membrane.

In another embodiment, the methods as described herein make use eukaryotic cells in the form of a coprecipitate with calcium phosphate. In another embodiment, DNA complexed with calcium phosphate is readily taken up and expressed by mammalian cell transfected by the methods provided herein. In another embodiment, DNA complexed with diethylamino-ethyl-dextran (DEAE-dextran) or DNA trapped in liposomes or erythrocyte ghosts is used in mammalian transformation. In another embodiment, bacterial protoplasts containing plasmids are fused to intact animal cells with the aid of chemical agents such as polyethylene glycol (PEG). In another embodiment, DNA is directly introduced into cells by microinjection.

In another embodiment, the invention further provides methods of generating hyperstable mRNA in plants. In another embodiment, generating hyperstable mRNA in plants comprises the introduction of DNA sequences by insertion into the transforming (T)-DNA region of the tumor-inducing (Ti) plasmid of Agrobacterium tumefaciens. In another embodiment, generating a hyperstable mRNA in plants comprises the introduction of DNA sequences in liposomes, as well as induction of DNA uptake in plant protoplasts. In another embodiment, DNA fragments of the invention are introduced into plant cells by electroporation. In another embodiment, DNA fragments of the invention comprised within Plasmid DNA are introduced into plant cells by electroporation. In another embodiment, the methods of generating hyperstable mRNA in plants. Results in stably inherited and expressed desired gene.

In another embodiment, the DNA fragment encoding the hyperstable mRNA is inserted into a simian virus 40 (SV40) vector and a “helper” virus. In another embodiment, the DNA fragment encoding the hyperstable mRNA is introduced into animal cells by an Adeno-SV40 hybrid virus system.

In another embodiment, the DNA fragment encoding the hyperstable motif (stability inducing motif) in the mRNA molecule is a beta globin stability inducing motif. In another embodiment, the DNA fragment encoding the hyperstable motif comprises a hexnucleotide sequence within the 3′UTR mRNA molecule. In another embodiment, the DNA fragment encoding the hyperstable motif comprises two adjacent hexnucleotides sequences within the 3′UTR mRNA molecule. In another embodiment, the DNA fragment encoding the hyperstable motif comprises a nucleolin binding site. In another embodiment, nucleolin is the major nucleolar protein of growing eukaryotic cells. In another embodiment, nucleolin is found associated with intranucleolar chromatin and preribosomal particles. In another embodiment, nucleolin induces chromatin decondensation by binding to histone H1. In another embodiment, nucleolin further interacts with APTX and/or NSUN2. In another embodiment, nucleolin is a component of the SWAP complex that consists of NPM1, NCL/nucleolin, PARP1 and SWAP70. In another embodiment, nucleolin is a component of a complex which is at least composed of HTATSF1/Tat-SF1, the P-TEFb complex components CDK9 and CCNT1, RNA polymerase II, SUPT5H, and NCL/nucleolin. In another embodiment, nucleolin binding site is a nucleolin beta-globin binding site.

In another embodiment, the mRNA molecule is a mRNA molecule comprising a desired gene. In another embodiment, the mRNA molecule is a mRNA molecule comprising a stability inducing motif and a desired gene. In another embodiment, the mRNA is an exogenous mRNA thus the source of the desired gene and the recipient cell differ. In another embodiment, the desired gene is further manipulated by inducing specific mutations. In another embodiment, the mutations comprise deletions. In another embodiment, the mutations comprise insertions.

In another embodiment, the mRNA encodes a transcription factor. In another embodiment, the mRNA encodes a basal transcription factor. In another embodiment, the mRNA encodes a hormone that regulates gene expression. In another embodiment, the hormone binds to a receptor to form a gene-specific factor. In another embodiment, the mRNA encodes a growth factors or homeotic proteins that act as gene-specific factors or form complexes that do. In another embodiment, the transcription factor is an activator. In another embodiment, the transcription factor is a repressor. In another embodiment, the transcription factor binds to the promoter outside of the TATA box, especially near the transcription initiation site, the beginning of the DNA sequence that is actually read by RNA polymerase. In another embodiment, the transcription factor binds to sequences within the coding region of the gene, or downstream from it at the termination region. In another embodiment, the transcription factor binds to DNA sequences hundreds or thousands of nucleotides away from the promoter. In another embodiment, the transcription factor interacts with the basal factors, altering the rate at which they bind to the promoter. In another embodiment, the transcription factor influences RNA polymerase's rate of escape from the promoter, or its return to it for another round of transcription.

In another embodiment, the transcription factor physically alters the local structure of the DNA, making it more or less accessible. In another embodiment, the transcription factor comprises a helix-turn-helix motif. In another embodiment, the transcription factor is a homeotic protein. In another embodiment, the transcription factor comprises a zinc-finger motif. In another embodiment, the transcription factor comprises a steroid receptor.

In another embodiment, the mRNA encodes a growth factor. In another embodiment, a growth factor comprises aAny of a group of biologically active poly-peptides which function as hormonelike regulatory signals, controlling the growth and differentiation of responsive cells.

In another embodiment, the growth factor is an insulin family growth factor comprising somatemedins A and C, insulin, insulinlike growth factor (IGF), and multiplication-stimulating factor (MSF).

In another embodiment, the growth factor is a sarcoma growth factor (SGF). In another embodiment, the growth factor is a transforming growth factor (TGF). In another embodiment, the growth factor is an epidermal growth factor (EGF). In another embodiment, the growth factor is a nerve growth factor (NGF). In another embodiment, the growth factor is a fibroblast growth factor (FGF). In another embodiment, the growth factor is a platelet-derived growth factor (PDGF).

In another embodiment, the mRNA encodes a signaling molecule. In another embodiment, the signaling molecule is a neurotransmitter.

In another embodiment, the invention further provides a method of increasing the amount of a mRNA molecule in a cell, comprising the step of inserting a stability inducing motif at the 3′UTR stem-loop structure, thereby increasing the amount of a mRNA molecule in a cell. In another embodiment, the method further comprises the step of increasing the expression rate of said mRNA molecule. In another embodiment, the step of inserting a stability inducing motif at the 3′UTR stem-loop structure does not increase the expression rate of said mRNA molecule. In another embodiment, increasing the stability of a mRNA molecule by inserting a stability inducing motif at the 3′UTR stem-loop structure and increasing the expression rate of the mRNA molecule, are two distinct molecular modifications leading to an increase in the amount of the mRNA molecule compared to a control sample. In another embodiment, a control sample comprises an unmodified-unstabilized mRNA molecule.

In another embodiment, increasing the expression rate of a mRNA molecule comprises manipulating a gene promoter element. In another embodiment, increasing the expression rate of a mRNA molecule comprises inserting an inducible promoter element. In another embodiment, increasing the expression rate of a mRNA molecule comprises inserting a constitutively active promoter element.

In another embodiment, the method of the invention provides at least 1.5 folds increase in the amount of a mRNA molecule in a cell. In another embodiment, the method of the invention provides at least 2 folds increase in the amount of a mRNA molecule in a cell. In another embodiment, the method of the invention provides at least 4 folds increase in the amount of a mRNA molecule in a cell. In another embodiment, the method of the invention provides at least 6 folds increase in the amount of a mRNA molecule in a cell. In another embodiment, the method of the invention provides at least 10 folds increase in the amount of a mRNA molecule in a cell.

In another embodiment, the method of the invention provides at least 20 folds increase in the amount of a mRNA molecule in a cell. In another embodiment, the method of the invention provides at least 30 folds increase in the amount of a mRNA molecule in a cell. In another embodiment, the method of the invention provides at least 40 folds increase in the amount of a mRNA molecule in a cell. In another embodiment, the method of the invention provides at least 50 folds increase in the amount of a mRNA molecule in a cell. In another embodiment, the method of the invention provides at least 60 folds increase in the amount of a mRNA molecule in a cell. In another embodiment, the method of the invention provides at least 80 folds increase in the amount of a mRNA molecule in a cell. In another embodiment, the method of the invention provides at least 100 folds increase in the amount of a mRNA molecule in a cell.

In another embodiment, the method of the invention provides at least 1.5 folds increase in the amount of protein translated from a mRNA molecule in a cell. In another embodiment, the method of the invention provides at least 2 folds increase in the amount of protein translated from a mRNA molecule in a cell. In another embodiment, the method of the invention provides at least 3 folds increase in the amount of protein translated from a mRNA molecule in a cell. In another embodiment, the method of the invention provides at least 4 folds increase in the amount of protein translated from a mRNA molecule in a cell. In another embodiment, the method of the invention provides at least 5 folds increase in the amount of protein translated from a mRNA molecule in a cell. In another embodiment, the method of the invention provides at least 6 folds increase in the amount of protein translated from a mRNA molecule in a cell. In another embodiment, the method of the invention provides at least 8 folds increase in the amount of protein translated from a mRNA molecule in a cell. In another embodiment, the method of the invention provides at least 10 folds increase in the amount of protein translated from a mRNA molecule in a cell.

In another embodiment, the method of the invention provides at least 20 folds increase in the amount of protein translated from a mRNA molecule in a cell. In another embodiment, the method of the invention provides at least 30 folds increase in the amount of protein translated from a mRNA molecule in a cell. In another embodiment, the method of the invention provides at least 40 folds increase in the amount of protein translated from a mRNA molecule in a cell. In another embodiment, the method of the invention provides at least 60 folds increase in the amount of protein translated from a mRNA molecule in a cell. In another embodiment, the method of the invention provides at least 80 folds increase in the amount of protein translated from a mRNA molecule in a cell. In another embodiment, the method of the invention provides at least 100 folds increase in the amount of protein translated from a mRNA molecule in a cell.

In another embodiment, the method of the invention provides that increasing the stability of a mRNA molecule correlated to the amount of a protein translated from a mRNA molecule. In another embodiment, the method of the invention provides that increasing the stability of a mRNA molecule comprises increasing the amount of protein translated therefrom.

In another embodiment, the invention further provides a method of producing an exogenous protein in a eukaryotic cell, comprising the step of inserting a stability inducing motif at the 3′UTR stem-loop structure of a mRNA molecule encoding a protein, thereby producing an exogenous protein in a eukaryotic cell. In another embodiment, the method further comprises the step of increasing the expression rate of a mRNA molecule.

### Experimental Details Section

### Materials and Methods

### Cell Culture

HeLa cells expressing the tetracycline-regulated transactivator (tTA) fusion protein (BD Biosciences) were maintained in Dulbecco's modified Eagle's medium supplemented with 10% fetal bovine serum in a humidified 5% CO₂ environment. Suspension MEL cells were cultured under similar conditions, while human K562 cells were grown in Iscove's modified Dulbecco's medium containing 4 mM glutamine and 1.5 g/liter sodium bicarbonate and supplemented with 10% fetal bovine serum. Cells (˜5×10⁵) were transfected with 5 μg supercoiled DNA using Superfect reagent as recommended by the manufacturer (QIAGEN). Doxycycline was added to a final concentration of 1 μg/ml when required.

### Gene Cloning

pTRE-beta^{WT} was constructed from a 3.3-kb fragment of human genomic DNA containing the intact beta-globin gene and contiguous 3′ flanking region, inserted into the SacII-ClaI polylinker site of pTRE2 (BD Biosciences). Linker-scanning mutations were introduced into the human beta-globin gene by a splice overlap extension-PCR method using paired, complementary 30-nt primers containing the desired HindIII mutation (5′AAGCTT3′). The resulting mutated 904-bp cDNAs were then substituted for the cognate EcoRIEcoNI fragment of pTRE-beta^{WT}. Chemically competent DH5alpha Escherichia coli cells were transformed (Invitrogen), mini-prep DNA was prepared from individual colonies (QIAGEN), and the structures of the variant beta-globin genes were subsequently validated by HindIII digestion and by automated dideoxy sequencing. pTRE-beta^{ARE104} and pTRE-beta^{ARE130} were constructed by introducing a 59-bp A/U-rich mRNA instability element into the HindIII sites of pTRE-beta^{ARE104} and pTRE-beta^{ARE130}, respectively.

### RNase Protection Analysis

Cellular RNAs prepared from cultured cells using TRIzol reagent (Gibco-BRL) were analyzed as described previously. ³²P-labeled beta-globin and beta-actin probes were prepared by in vitro transcription of DNA templates using SP6 RNA polymerase (Ambion). The 287-nt beta-globin probe protects a 199-nt sequence of human beta-globin mRNA exon II, while the 313-nt beta-actin probe protects a 160-nt exonic fragment of human beta-actin mRNA. Band intensities were quantitated from PhosphorImager files using Image-Quant software (Amersham Biosciences).

### RT-PCR⁺¹ Analysis

Purified RNAs (−500 ng) were reverse transcribed and thermally amplified using Superscript one-step reagents under conditions recommended by the manufacturer (Invitrogen) and then amplified for 40 cycles using exon II (5′ACCTGGACAACCTCAAGG3′) and exon III (5′TTTTTTTTTTGCAATGAAAATAAATG3′) primers that generate a 355-bp cDNA product encompassing the full beta-globin 3′UTR. Reaction mixtures were subsequently augmented with 100 mmol of a nested ³²P-labeled exon II primer (5′CCACACTGAGTGAGCTGC3′) and 0.5 μl Platinum Taq (Invitrogen) and product DNA amplified for one additional cycle. This method generates 328-nt ³²P-labeled homodimeric DNAs that fully digest with HindIII to generate ³²P-labeled products between 189 and 285 bp in length.

### Proteomics

Proteomics Facility. Tryptic digests were resolved on a Voyager DE Pro (Applied Biosystems), and protein identities were deduced from MS-Fit (University of California) analysis of peptide fragments using the NCBInr database. Time-of-flight (TOF)-TOF analysis was carried out using a 4700 proteomics analyzer (Applied Biosystems) equipped with Global Proteomics Server analytical software.

### Cytosolic Extract

Briefly, phosphate-buffered saline (PBS)-washed cells were incubated for 20 mM at 4° C. in RNA immunoprecipitation assay (RIPA) buffer (50 mM Tris-HCl [pH=7.4], 150 mM NaCl, 1 mM EDTA, 1% NP-40, 1 mM Na3VO4, 1 mM NaF, and 1× protease inhibitor cocktail [BD Biosciences]). The lysate was centrifuged at 13,000×g for 15 mM, and the supernatant was collected and stored at −80° C. For cross-linking studies, in vitro-transcribed, ³²P-labeled RNAs were incubated with cytoplasmic extract and exposed to UV light (3,000 mJ/cm2) for 5 min

### Fluorescence-Activated Cell Sorter (FACs) Analysis

EDTA-anticoagulated whole blood was stained with thiazole orange as directed by the manufacturer (Sigma). Erythroid cells were identified by their characteristic forward- and sidescatter properties using a FACSVantage cell sorter equipped with Digital Vantage options (Becton-Dickinson). Thiazole orange-staining cells (reticulocytes) were collected, excluding a small population of hyper-staining nucleated erythroid progenitor cells.

### Affinity Enrichment Studies

Custom 5′-terminal biotinylated single-stranded DNAs (ssDNAs) were purchased from Integrated DNA Technologies (Coralville, Iowa). Molar equivalents of each ssDNA (3 μmol) were incubated for 1 h at 4° C. in PBS (pH 7.2) along with 100 μl of preequilibrated ImmunoPure immobilized avidin agarose beads (Pierce Biotechnology). The pelleted beads were washed four times with PBS, incubated at 4° C. for 1 h with 1 ml cytoplasmic extract, and then washed five times with PBS. Bound proteins were eluted with loading buffer and resolved on precast 4 to 12% gradient sodium dodecyl sulfatepolyacrylamide gel electrophoresis (SDS-PAGE) gels as recommended by the manufacturer (Invitrogen). A parental ssDNA corresponding to the beta-globin 3′UTR stem-loop structure (5′ATTTCTATTAAAGGTTCCTTTGTTCCCTAAGTCCAACTACTAAACTGGGGGATATTATG AAGGGCCTTGAGCATC3′ (SEQ ID No. 4)) was modified by the deletion of an internal 18-nt sequence (5′GGGGGATATTATGAAGGG3′, SEQ ID No. 5) and by the substitution of an unrelated 18-nt sequence (5′ATGCCGTAATGCCGTAAT3′, SEQ ID No. 7) or a sequence encompassing the beta-PRE (5′TTCCTTTGTTCCCTAAGT3′ (SEQ ID No. 6) at the same site.

### Western Blotting

Antibodies purchased from Santa Cruz Biotechnology included mouse monoclonal anti-human nucleolin (MS-3), rabbit polyclonal antihuman nucleolin (H-250), goat polyclonal anti-human HDAC-2 (C-19), rabbit polyclonal anti-human tumor necrosis factor alpha, and goat polyclonal antihuman hnRNP-E1 (T-18). Rabbit polyclonal anti-human actin antibodies were purchased from Sigma (A-2066). Protein samples in loading buffer were denatured at 100° C. for 5 min, resolved on a precast 4 to 12% gradient SDS-PAGE gel, and transferred to a nitrocellulose membrane using an XCell II blot module according to the manufacturer's instructions (Invitrogen). Blots were blocked for 1 h at room temperature in PBS containing 0.1% Tween 20, supplemented with 3% dried milk, and then incubated for an additional hour following antibody addition. Membranes washed with the Tween 20-PBS mixture were subsequently incubated for 1 h with a horseradish peroxidase-conjugated secondary antibody (Amersham Biosciences) and analyzed using a chemiluminescence method (ECL kit; Amersham).

### RNA Immunoprecipitation

HeLa cell extracts were prepared. PBS-washed erythrocytes were isolated from EDTA-anticoagulated whole blood by fractionation over a Histopaque 1.077/1.119 bilayer cushion (Sigma). Extracts prepared in RIPA buffer (1 ml) were precleared with 60 μl protein A-agarose beads (Invitrogen) and then incubated at 4° C. for 3 h with nucleolin H-250 antibodies. Fresh protein A-agarose beads (60 μl) were then added, and the incubation continued for another 2 h. Immunoprecipitates were washed three times in RIPA buffer, and bound RNAs were collected by TRIzol extraction and ethanol precipitation for subsequent analysis. Control 18S pre-RNAs were RT-PCR amplified using oligomers 5′GTTCGTGCGACGTGTGGCGTGG3′ and 5′CAGACCCGCGACGCTTCTTCGT3′, producing a 501-bp cDNA fragment.

### Preparation of Recombinant Alpha-CP and Purification of Nucleolin

A glutathione S-transferase alpha-CP1 fusion protein was purified from DHSalpha cells transfected with pEGX-6P-alpha-CP1 (kind gift of M. Kiledjian, Rutgers University); the glutathione S-transferase domain was subsequently cleaved with PreScission proteinase (Pharmacia Biotech). Human nucleolin was affinity enriched from HeLa and/or K562 cell extract using an agarose-immobilized 2′-O-methyl RNA sequence (5′UAUUAAAGGUUCCUUUGUUCCCUAAGUCCAAC3′). A related method was used to prepare nucleolin-depleted extract.

### Example 1

### Validation of a Method for Analyzing the Stability of Beta-Globin mRNA in Intact Cells

To facilitate the studies of beta globin mRNA stability, a system in which a single defined gene can be transcriptionally silenced in intact, translationally competent cells was developed. This approach permits mRNA decay to be assessed in vivo using a transcriptional chase approach that does not compromise cell viability. The method requires cells that constitutively express a tTA fusion protein that activates genes linked to a recombinant hybrid tetracycline response element (TRE). tTA activity is rapidly and efficiently inhibited in the presence of tetracycline or doxycycline (Dox), which does not affect the expression of other, constitutively expressed eukaryotic genes. Consequently, the stabilities of mRNAs encoded by TRE-linked genes can be estimated by assessing their rate of disappearance from Dox-treated cells. The proposed use of tTA-expressing HeLa cells was tested by assessing the fate of mRNAs carrying a known mRNA destabilizing determinant, the 3′UTR A/U-rich element (ARE) derived from human granulocyte-macrophage colony-stimulating factor mRNA (70) (FIG. 1A). TRE-linked beta-globin geneswere constructed to contain either the native 3′UTR (pTRE-BETA^{WT}) or 3′UTRs engineered to contain single-copy ARE inserts (pTRE-beta^{ARE104} and pTRE) beta^{ARE130}. pTRE-beta was cotransfected into HeLatTA cells with either pTRE-beta^{ARE104} or pTRE-beta^{ARE130}, and the levels of their encoded mRNAs were established at defined intervals following Dox exposure. Unlike with beta^{WT} mRNA, the level of each beta^{ARE} mRNA fell rapidly (FIGS. 1B and C), confirming the utility of the tTA-TRE system for differentiating unstable and stable mRNAs in intact, cultured cells.

### Example 2

### Human Beta-Globin mRNA is Destabilized by Either of Two Adjacent Site-Specific 3′UTR Mutations

To map critical cis determinants of beta-globin mRNA stability, 17 full-length beta-globin genes were constructed, each containing a hexanucleotide substitution at a unique 3′UTR position (FIG. 2A). Collectively, the mutations saturate 102 nt of the 107-nt sequence of beta-globin 3′UTR between the native TAA translational termination codon and the AATAAA polyadenylation signal. He-LatTA cells were cotransfected with DNA mixes comprising different combinations of TRE-linked, variant-globin genes, including one)(beta^{H100} that was arbitrarily selected as an internal control (FIG. 2B). The level of each variant beta^{H} mRNA, relative to that of beta^{H100} mRNA, was subsequently determined by RT-PCR⁺¹ following 24- and 48-hour exposures to Dox. Two of the variant beta^{H} mRNAs containing hexanucleotide substitutions at 3′UTR positions 122 and 124 displayed levels that fell four- to fivefold faster than those of other variant beta^{H} mRNAs (FIGS. 2C and D). These results were confirmed in a duplicate analysis utilizing a different post-Dox interval (not shown) and in related experiments in which genes encoding unstable variant beta^{H122} and beta^{H124} mRNAs and stable variant beta^{H114} mRNA were separately transfected into HeLatTA cells along with internal control pTRE-beta^{H100} FIG. 2E). Formal mRNA stability studies were subsequently carried out using Dox-exposed HeLatTA cells that had been cotransfected with TRE-linked genes encoding beta^{WT} and either beta^{H114} or beta^{H124} mRNA (FIG. 2F to H). By comparison to the level of beta^{WT} mRNA, that of beta^{H124} mRNA fell rapidly (FIGS. 2F and H), while that of control beta^{H114} mRNA remained stable (FIGS. 2G and H).

The combined results of screening and formal mRNA stability analyses confirm the importance of the 12-nt H122/H124 sequence to the intrinsically high stability of beta-globin mRNA.

### Example 3

### Nucleolin Binds to the Beta-Globin 3′UTR in Intact Cultured Cells and Primary Erythroid Cells

The stabilities of many mRNAs, including those encoding beta-globin, alpha 1(I) collagen (73), tyrosine hydroxylase, histone, and the transferring receptor, require the assembly of defined mRNP effector complexes on specific determinants within their 3′UTRs.

To identify candidate trans-acting factors that might functionally interact with the beta-globin 3′ UTR, agarose-immobilized ssDNAs corresponding to the beta^{WT} 3′UTR and to negative control poly(dI·dC) were separately incubated with cytoplasmic extract prepared from cultured human erythroid K562 cells. Three bands that displayed relative specificities for the beta^{WT} 3′UTR were subsequently excised and subjected to matrix-assisted laser desorption ionization (MALDI)-TOF analysis (FIG. 3A). The ˜100-kDa band was unambiguously identified as nucleolin from 14 tryptic peptide fragments representing 22% coverage (molecular weight search, 1.469×10⁴) (FIG. 3B); the identities of the remaining two bands could not be established with certainty. Companion experiments indicated that nucleolin binds equally well to related full-length and truncated agarose-immobilized RNAs and 2′-O-methylated RNAs, respectively (FIG. 3C). These results were corroborated by parallel TOF-TOF analyses of affinity-enriched erythroid MEL cell extract that also unequivocally identified nucleolin (data not shown). This dual preliminary identification was subsequently confirmed by Western blot analysis of affinity-enriched proteins using a polyclonal nucleolin antibody (FIG. 3D). Nucleolin appears to bind to the beta-globin 3′UTR in a sequence-specific manner, as increasing quantities of an unrelated soluble competitor ssDNA effectively compete background proteins from an agarose-immobilized ssDNA beta-globin 3′UTR ligand but do not affect nucleolin binding (FIG. 3E). In addition, UV-cross-linked nucleolin-beta-3′UTR mRNPs assemble in K562 cytoplasmic extract but not in extracts that are affinity depleted of nucleolin, confirming that nucleolin also binds to beta^{WT} RNA (FIG. 3F, lanes T and D, respectively). These results document the sequence-specific binding of nucleolin to the beta-globin mRNA 3′UTR in vitro and suggest that this interaction may subserve a critical function in vivo.

### Example 4

### Nucleolin Localizes to the Cytoplasm of Intact Erythroid Progenitor Cells

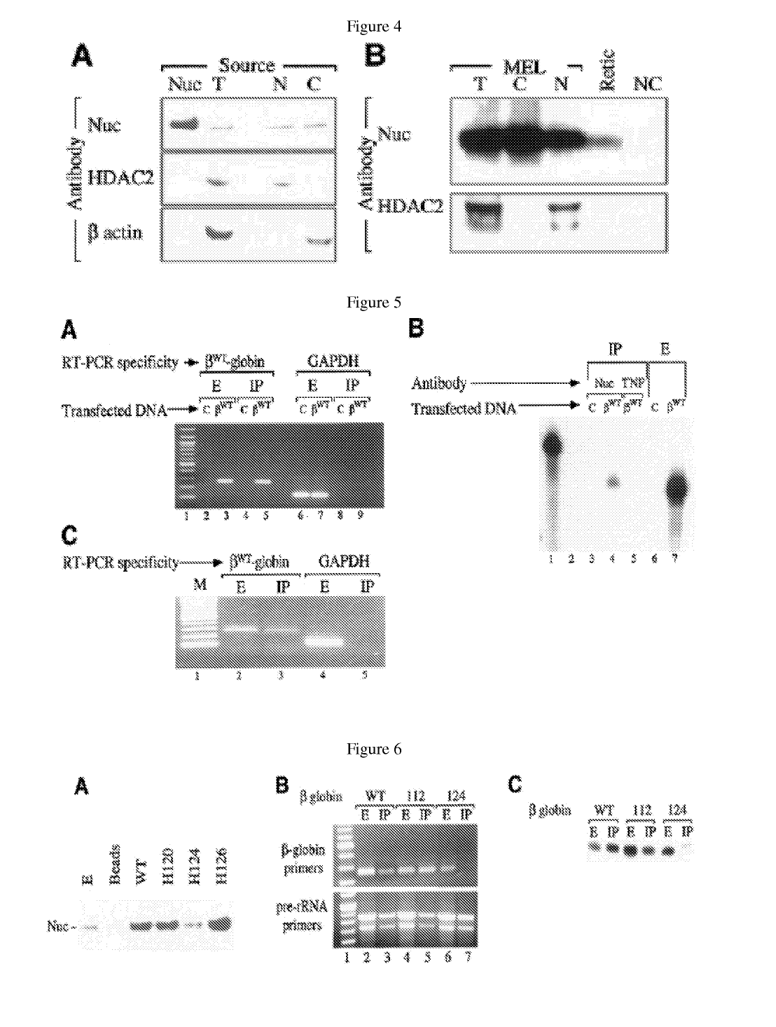

Although nucleolin has been identified in the cytoplasm of nonerythroid cells, its presence in erythroid cytoplasm has never been formally established. Two methodologically independent approaches were used to demonstrate that nucleolin can be found in the cytoplasm of erythroid cells representing temporally distinct stages of terminal differentiation. Nucleolin was easily detected by Western analysis of cytoplasm prepared from murine erythroid MEL cells (FIG. 4A) and was also identified in extract prepared from FACS-sorted murine reticulocytes (FIG. 4B). These results con-firm that nucleolin is abundant in erythroid cytoplasm, permitting consideration of its potential role in stabilizing the relatively ure population of globin mRNAs that also populate these cells.

### Example 5

### Nucleolin Binds Human Beta-Globin mRNA in both Cultured Cells and Primary Human Erythroid Progenitors

The demonstration that nucleolin binds to ssDNA and RNA corresponding to the beta-globin 3′UTR in vitro predicted its capacity to interact with full-length beta-globin mRNA transcripts in vivo in intact cells. This hypothesis was subsequently tested using an RNA-immunoprecipitation (RIP) method. Human beta-globin mRNA was detected in cell extract as well as in a nucleolin immunoprecipitate prepared from cells transfected with pTRE-beta^{WT} (FIG. 5A, lanes 3 and 5) but not in fractions prepared from cells transfected with an empty pTRE control vector (lanes 2 and 4). The specificity of the nucleolin-globin mRNA interaction was indicated by control experiments in which constitutively expressed GAPDH (glyceraldehyde-3-phosphate dehydrogenase) mRNA was observed in cell extract (FIG. 5A, lanes 6 and 7) but not in the nucleolin immunoprecipitate (FIG. 5A, lanes 8 and 9). Human beta-globin mRNA was not identified in immunoprecipitate prepared with an unrelated antibody (FIG. 5B, compare lanes 4 and 5), demonstrating that the results do not arise from artifactual binding of beta-globin mRNA to immunoglobulin. The likely physiological importance of the interaction between nucleolin and the beta-globin mRNA was indicated by RIP analyses of lysate prepared from density-fractionated human erythroid progenitors. Both beta-globin mRNA and control GAPDH mRNA were observed in the unfractionated lysate (FIG. 5C, lanes 2 and 4), while beta-globin mRNA, but not GAPDH mRNA, was detected in immunoprecipitate prepared using nucleolin antibody (FIG. 5C, compare lanes 3 and 5). These experiments confirm that beta-globin mRNA and nucleolin interact with high mutual specificity in intact cultured cells as well as in primary human erythrocytes.

### Example 6

### An mRNA-destabilizing mutation in the beta-globin 3′UTR Reduces Nucleolin Binding In Vitro and In Vivo

The proposed functional linkage between nucleolin binding and beta-globin mRNA stability was subsequently investigated by assessing the affinity of nucleolin for variant beta^{H}-globin mRNAs containing destabilizing and control nondestabilizing 3′UTR hexanucleotide linker-scanning substitutions. The affinity of purified nucleolin for ssDNAs corresponding to the beta-globin 3′UTR was substantially reduced by the mRNA-destabilizing H124 mutation but not by flanking mutations at position H120 or H126 that had had no discernible effect on beta-globin mRNA stability in earlier in vivo studies (FIG. 6A). The adverse effect of the H124 mutation on nucleolin binding was also demonstrated in vivo using RIP analyses of HeLatTA cells expressing beta^{WT} beta^{H112} and beta^{H124} mRNAs (FIG. 6B). Each mRNA was easily detected in the cell extract (FIG. 6B, lanes 2, 4, and 6), while only the stable beta^{WT} and beta¹¹² mRNAs—but not the unstable beta¹²⁴ mRNA—were present in the nucleolin immunoprecipitate (FIG. 6B, lanes 3, 5, and 7). Pre-rRNA, which is known to bind nucleolin strongly (1), was observed in all samples, confirming the quality of the mRNAs and controlling for other aspects of the experimental method. These results were corroborated by parallel analyses of beta^{WT}, beta¹¹², and beta¹²⁴ mRNAs using an independent RNase protection approach (FIG. 6C) and confirmed in repeat analyses (data not shown). Consequently, the native sequence targeted by the H124 mutation appears to function both as a determinant of beta-globin mRNA stability and as a binding site for nucleolin, providing a critical link between these two processes.

This FIG. 6. Differential binding of nucleolin to mRNA-stabilizing and -destabilizing 3′UTR determinants. (A) beta-globin mRNA-destabilizing linker-scanning mutations reduce nucleolin binding in vitro. Agarose-immobilized, 59-nt ssDNAs corresponding to the proposed 3′UTR nucleolinbinding region of beta-globin mRNA were incubated in cytoplasmic extract, and adherent proteins were assessed by Western transfer analysis using nucleolin antibody. The wild-type sequence (WT) as well as sequences containing destabilizing (H124) and nondestabilizing (H120 and H126) HindIII mutations were assessed. Unfractionated extract (E) and extract adhering to unliganded agarose beads were run in the first two lanes as controls. (B, C) Full-length, unstable H124 mRNA binds nucleolin poorly in vivo in intact, cultured cells. Unfractionated cell extract or nucleolin immunoprecipitate (IP) was prepared from cultured cells transfected with genes encoding beta^{WT}, beta^{H112}, and beta^{H124} mRNAs. (B) Recovered RNAs were RT-PCR amplified using primers specific to beta-globin mRNA (top) or to internal control pre-rRNA (bottom). The reaction products were resolved on an ethidium bromide-stained, nondenaturing polyacrylamide gel. Lane 1 contains a 100-bp DNA ladder. (C) Recovered RNAs were assessed by RNase protection using an in vitro-transcribed, 32P-labeled beta-globin RNA probe.

### Example 7

### A Model for Beta-Globin mRNA Stability

Although the beta-PRE appears to be a determinant of beta-globin mRNA stability in vivo, its anticipated role as a target for alpha-CP (αCP) binding has been difficult to recapitulate in vitro. A model for beta-globin mRNA stability is proposed, which incorporates the findings presented here and, in addition, accounts for previous experimental evidence that indirectly implicates αCP in this process. In this model, the beta-globin 3′UTR has the potential to assume a highly stable stem-loop structure that incorporates the β-PRE and nucleolin-binding sites into its left and right half-stems, respectively (FIG. 7A). If secondary structure were to inhibit the access of αCP to the β-PRE-binding site, then any process that weakens the stem structure would be predicted to facilitate αCP binding (FIG. 7B). The possibility that native secondary structure inhibits αCP binding was tested in three independent affinity-binding studies. Results from the first study suggest that αCP access to the β-PRE is highly dependent upon its mRNA context: recombinant αCP (r αCP) binds poorly to an ssDNA corresponding to the full-length β-3′UTR (FIG. 7C, lane 5), while binding avidly to ssDNAs corresponding to the β-PRE either in isolation (FIG. 7C, lane 6) or when inserted into a different 3′UTR (FIG. 7C, lane 7). In a second study, baseline interaction of r αCP with the left-half-stem β-PRE was ablated by its pre-incubation with an ssDNA corresponding to the right half-stem (FIG. 7D).

A third study demonstrated that αCP binds poorly to the intact 3′UTR stem-loop structure (FIG. 7E, lane 2) while, in agreement with predictions, binding strongly to 3′ UTRs that contain stem-destabilizing substitutions (FIG. 7E, lanes 3 and 6) or deletions (FIG. 7E, lane 5). The results of all three experiments are consistent with a model in which native structure within the beta-globin 3′UTR must be remodeled as a precondition for αCP interaction with the β-PRE. The potential role that nucleolin may play in remodeling the 3′UTR stem-loop structure in vivo was investigated by assessing the binding of r αCP to agarose-immobilized beta-globin 3′UTRs in vitro under different conditions. The poor baseline affinity of r αCP for the naked probe is significantly enhanced by preincubating the beta-globin 3′UTR with affinity-purified nucleolin (FIG. 7F, compare lanes 2 and 4). Although this result does not favor any specific mechanism, the possibility that nucleolin facilitates αCP binding through its effect on mRNA FIG. 4. Nucleolin is present in the cytoplasms of differentiating erythroid cells. (A) Nucleated erythroid progenitors contain cytoplasmic nucleolin. Western blot analysis was performed on total (T), nuclear (N), and cytoplasmic (C) extracts prepared from MEL cells using nucleolin (Nuc) antibody. The blot was stripped and rehybridized with antibodies directed against nucleus- and cytoplasm-specific histone deacetylase-2 (HDAC-2) and a actin, respectively. Affinity-purified nucleolin was analyzed in parallel as a positive control. (B) Anucleate erythroid progenitors (reticulocytes) contain cytoplasmic nucleolin. Hemolysate prepared from FACS-sorted murine reticulocytes (Retic) was analyzed by Western transfer analysis using nucleolin antibody.

Total, cytoplasmic, and nuclear extracts prepared from MEL cells were analyzed in parallel as positive controls, and recombinant αCP was run as a negative control (NC). The blot was stripped and rehybridized with HDAC-2 antibody to confirm the absence of contaminating nucleoplasm in the Retic sample.

FIG. 5. Nucleolin binds to beta-globin mRNA in intact cells. (A, B) Specificity of nucleolin-beta-globin mRNA interaction in vivo. (A) HeLatTA cells were transfected with pTRE-β^{WT} (β^{WT}) or with an empty pTRE vector control (C). Total RNA recovered from cell extract (E) or nucleolin immunoprecipitate (IP) was RT-PCR amplified using β^{WT} sequence-specific oligomers, generating a 261-bp product (lanes 2 to 5), or with GAPDH mRNA-specific oligomers, producing a 116-bp product (lanes 6 to 9). Lane 1 contains a 100-bp DNA ladder. (B) Total RNA was recovered from immunoprecipitate (lanes 3 to 5) or extract (lanes 6 and 7) prepared from cells transfected with pTRE-β^{WT} or with the empty pTRE vector control (C) Immunoprecipitates were prepared using nucleolin- or tumor necrosis factor-specific antibodies (Nuc or TNF, respectively). RNAs were analyzed by RNase protection using in vitro-transcribed, 32P-labeled RNA probes (84). Intact and RNase-digested 32P-labeled probes were run in lanes 1 and 2, respectively. (C) Nucleolin binds beta-globin mRNA in intact human erythroid cells. Purified RNA prepared from the extract or nucleolin immunoprecipitate of density-fractionated human erythroid cells was RT-PCR amplified using human β-globin- and GAPDH-specific oligomers. M, DNA size markers.

Downloaded from structure is suggested by the observation that αCP binding is also enhanced, in the absence of nucleolin, by prior heat denaturation of the agarose-immobilized β-3′UTR ligand (FIG. 7F, lane 3). In the aggregate, the results of these in vitro analyses are consistent with the assembly of a stable structure within the beta-globin 3′UTR that inhibits alpha-CP binding and suggest that nucleolin facilitates αCP access through interaction with this structure.

### Example 8

### A Model for Beta-Globin mRNA Stability

The normal expression of human alpha- and beta-globin proteins is critically dependent upon the high stabilities of their encoding mRNAs. The highly stable globin messages are selectively enriched in terminally differentiating erythroid cells, in contrast to non-globin mRNAs with substantially shorter half-lives. These cells are transcriptionally silenced, but remain translationally active, so that the abundant globin mRNAs produce high levels of a relatively pure population of globin protein.

The stability of b-globin mRNA in erythroid cells is regulated by two distinct elements within its 3′-untranslated region (3′UTR). This baseline stability might be enhanced by the substitution, deletion, or duplication of one or both regions. Such ‘hyperstable’ b-globin mRNAs would be expected to accumulate to high levels, increasing the expression of beta globin from therapeutic transgenes that have previously been transcriptionally optimized. These transgenes would be of great importance for the treatment of sickle cell disease and b-thalassemia.

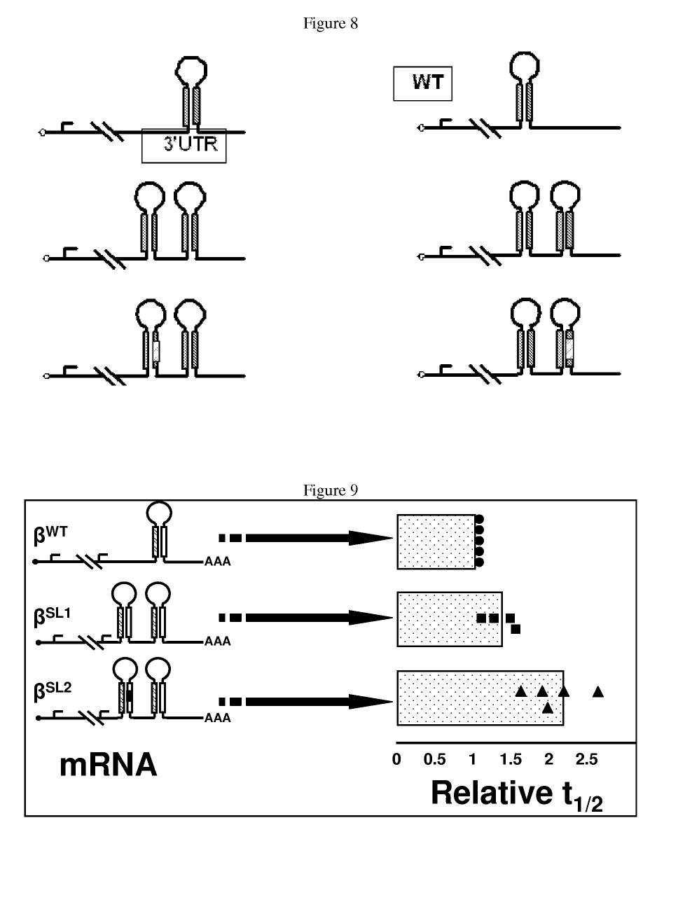

A secondary stem-loop structure exists within the beta-globin 3′UTR. beta-PRE is located on the left half-stem, while a stability element has been mapped to the right half-stem of the highly stable stem-loop structure, immediately opposite the beta-PRE. A stylized structure to the right illustrates the stability element is shown in FIGS. 7A, 8 and 9.

Using a saturation mutagenesis approach, genes that encoded the wild-type human beta-globin mRNA, as well as additional variant b-globin genes encoding β-globin mRNAs were constructed with site-specific hexanucleotide substitutions within their 3′UTRs. The structures of these genes were subsequently confirmed by dideoxy sequencing and restriction digest analysis.

The strategy capitalized on a novel cultured cell method in which a gene of interest is linked to a promoter element that binds a transcriptional transactivator that is constitutively active but that is inhibited in the presence of tetracycline or docycycline. This system permitted to determine the stability of WT and variant b-globin mRNAs in situ in intact cells using a transcriptional chase approach. The level of each variant beta-globin mRNA was assessed at defined time points following transcriptional silencing with tetracycline, relative to a control mRNA.

RT quantitative PCR method using Taqman probes specific for beta globin (gene of interest, and beta actin (edogenous control).

### Example 9

### Construction of Tetracycline-Conditional Genes Encoding Wild-Type and Variant Beta-Globin mRNAs with Site-Specific Mutations in their 3′UTRs

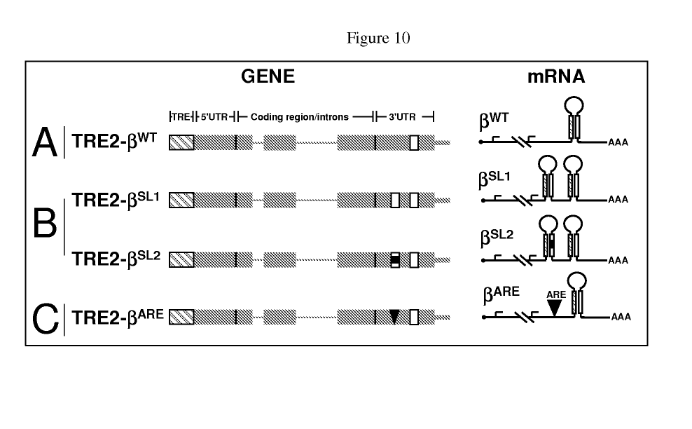

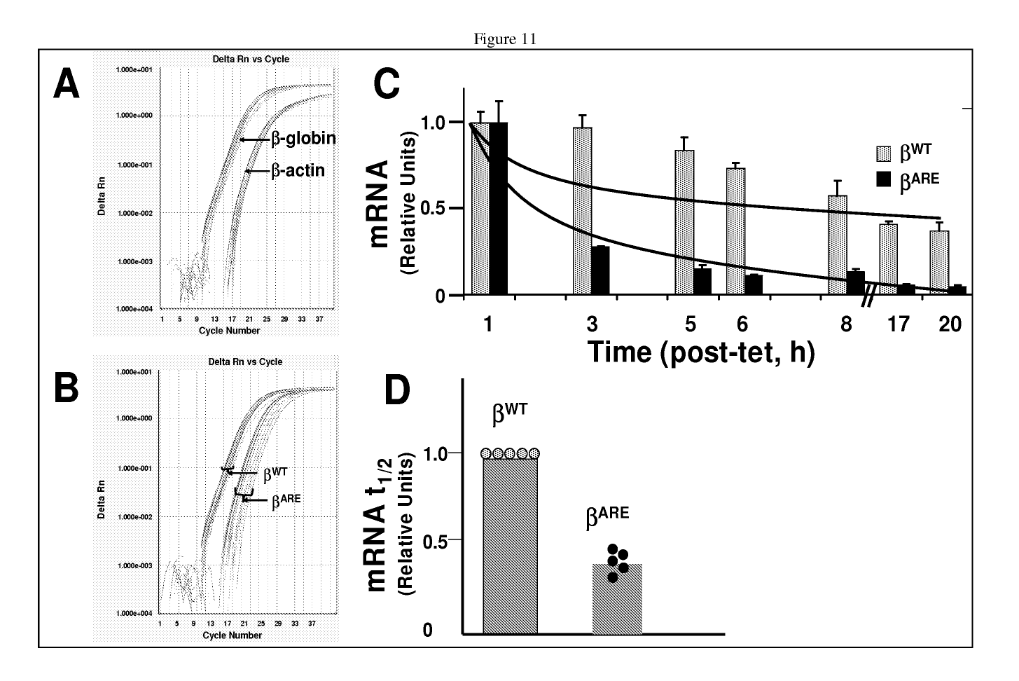

Previous examples indicate that the constitutive stability of beta-globin mRNA is determined, in part, by a stem-loop (SL) structure within its 3′UTR. Among several potential mechanisms, the SL structure may act to increase mRNA stability through a dominant positive effect. This mechanism would raise the possibility that replication of the SL motif, in the context of the intact 3′UTR, might further enhance the stability of human beta-globin mRNA. To test this hypothesis, four Tet-conditional genes encoding wild-type beta-globin mRNA or variant beta-globin mRNAs containing site-specific mutations in their 3′UTRs (FIG. 10A) were constructed. The structures of all genes were validated by restriction digest, as well as automated dideoxy sequencing of critical 3′UTR structures.

All four test genes were derived from the parental pTRE2 vector (Clontech) which contains a TRE promoter element followed by a multiple cloning site (MCS). pTRE2-β^{WT}, expressing the full-length human beta-globin mRNA, was generated by inserting a 3.3-kb fragment of human genomic DNA, containing the intact β-globin gene and contiguous 3′-flanking region, into the SacII-ClaI polylinker site of pTRE2.

The pTRE2-β^{WT} gene was further modified in two critical ways. First, a 1.2-kb vector sequence was deleted that provided an alternate site for 3′-cleavage/polyadenylation of the nascent mRNA transcript. Second, a 1.5-kb fragment of DNA containing the hygromycin-resistant gene, excised from a parental pTRE2hyg vector, was inserted into the vector XhoI site of pTRE2-β^{WT}. This modification was made in anticipation of generating cell lines that stably express TRE-linked genes encoding wild-type and variant beta-globin mRNAs in Aim IA. pTRE2-based plasmids encoding variant β-globin mRNAs with double-SL motifs were generated using a similar approach. A full-length human beta-globin gene containing a HindIII site at position 15 of its 3′UTR was inserted into the parental pTRE-2 vector as described above. Two 66-bp double-strand DNA fragments corresponding to the native beta-globin SL structure, or to a second, related SL structure containing a modification to the right half-stem, were commercially synthesized. The two DNAs were inserted into □-globin genes containing the position-15 HindIII mutation, generating two different beta-globin gene variants (pTRE2-β^{SL1} and -β^{SL2}) each containing a tandem motif within their 3′UTRs. A similar approach was used to construct a control gene (pTRE2-β^{ARE}) encoding a β-globin mRNA with a 59-bp A/U-rich instability element (ARE) at the position-15 HindIII site of the 3′UTR (FIG. 2C). The four gene constructs are referred to as β^{WT}, β^{SL1}, β^{SL2} and β^{ARE} for clarity.

### Example 10

### K562 Cells that Stably Express the Tetracycline-Regulated tTA Transactivator Protein

A suitable K562 cultured cell line expressing the tTA transactivator facilitates tight transcriptional regulation of transfected beta-globin genes and allows for high-level expression of the cognate beta-globin protein, properties that are critical. Cells were maintained in RPMI 1640 supplemented with 10% FBS and display a doubling time of approximately 24 hours. Cells are exposed to 30 μg/mL G418 weekly to ensure that the linked transfected tTA gene is not lost.

A sufficient number of low passage-number aliquots are stored under liquid N₂ for use in the proposed studies. Preliminary studies have been conducted in the applicant laboratory to demonstrate the absence of endogenous □-globin mRNAs and proteins that may interfere with the proposed studies.

### Example 11

### Stability Analyses of Variant □-Globin mRNAs Containing Site-Specific Duplication of the Stem-Loop Motif

Two complex studies have been conducted to assess the stabilities of variant beta-globin mRNAs in erythroid cells using tet-conditional K562^{tTA} cells. The first study establishes and validates a method for real-time quantitative RT-PCR (qRT-PCR) that is used to assess the relative levels of transiently expressed wild-type and variant beta-globin mRNAs in intact cultured cells. This study also demonstrates that the system is capable of distinguishing the difference in stability between wild-type beta-globin mRNA and a variant beta-globin mRNA that contains a known mRNA-destabilizing element within its 3′UTR.

A second study utilizes this method to assess the stabilities of beta-globin mRNAs containing two tandem SL structures within their 3′UTRs, demonstrating that their constitutive stability can be enhanced by duplicating the 3′UTR SL motif (see FIG. 11).

### Example 12

### qRT-PCR Method has been Established for Reproducible, High-Throughput Quantitation of Wild-Type and Variant □-Globin mRNAs

Consequently, a real-time RT-PCR method for assessing the decay of wild-type and variant □-globin mRNAs was designed and validated. The assay utilizes amplification/reporter Taqman probe sets for beta-globin mRNA that target the exon II/III sequence of beta-globin mRNA located proximal to its 3′UTR. This arrangement ensures that modifications in the 3′UTR will not affect either the binding efficiency of the probes or the processivity of DNA polymerase. Moreover, because the □-globin probe set bridges exons II and III, background signal from promiscuous amplification of genomic DNA is largely eliminated (RNA samples are pre-treated with DNase to further reduce this possibility).

The utility of the qRT-PCR method was validated in erythroid K562 cells that constitutively expressed the tTA transactivator protein (previous example). Cells were transfected with pTRE2-beta^{WT}, and aliqouts sacrificed at defined intervals following exposure to Tet. Levels of beta-globin mRNA in each aliquot were determined by qRT-PCR using the ΔΔCt method—a method for calculating relative mRNA quantities (RQ) by comparative Ct—, relative to internal control □-actin mRNA (FIG. 11A, 3B). The derivative □-globin mRNAs were expressed at high levels, as evidenced by the low cycle threshold (Ct) values. The condensed amplification curves indicate the narrow range of inter-sample variation. As predicted, beta-globin mRNAs containing the 59-nt ARE instability element, were rapidly degraded, by comparison to wild-type beta-globin mRNAs (FIG. 11C). Replicate analyses demonstrate that the calculated t_{1/2} value of wild-type beta-globin mRNA is nearly three times greater than that of the unstable control beta^{ARE} mRNA, indicating the high reproducibility of this novel assay (FIG. 11D). These studies confirm the suitability of the tTA-expressing K562 cells to distinguish stable and unstable mRNAs, as well as the qRT-PCR method to measure this effect.

### Example 13

### Transiently Expressed Beta-Globin mRNAs are Stabilized by the Addition of a Site-Specific SL Motif within their 3′UTRs

A proof-of-principle study was designed to test whether the stability of transiently expressed beta-globin mRNA could be enhanced by the addition of a site-specific SL motif within its 3′ UTR. K562^{tTA} cells were transiently transfected with TRE-linked genes encoding β^{WT}, β^{SL1} or β^{SL2} (generated as described previously), treated with Tet, and aliquots sacrificed at defined intervals thereafter. The level of beta-globin mRNA in each aliquot was determined by qRT-PCR relative to beta-actin mRNA, using the ΔΔCt method as described by Applied Biosystems (introduced in a previous example). Five replicate studies concur that the stabilities of mRNAs containing double-SL structures are increased between 1.5- and 2.5-fold, relative to wild-type beta-globin mRNAs carrying the single, native SL motif (FIG. 9). These findings clearly favor the principle that gain-of-function characteristics can be achieved by reasoned targeted site-specific mutagenesis.

Thus, a tetracycline-conditional method for assessing mRNA stability in erythroid K562^{tTA} was established, and was designed and constructed a unique TRE vector and several gene constructs encoding beta-globin and other test mRNAs, established and validated a reliable, sensitive and highly reproducible qRT-PCR analysis method; and importantly, confirmed by proof-of principle that the stability of beta-globin mRNA can be enhanced by specific introduced mutations within the 3′UTR. Collectively, these results provide substantial support for the hypothesis that mRNA stability can be manipulated.

The left of FIG. 9 represents the relative mRNA half lives of wild-type and two derivative beta globin constructs. Mean values from 4 or 5 separate experiments are reported. The left panel represents stylized structures of the WT construct (Top) and two different duplications of the stem-loop motif within the 3′ UTR. Analysis indicated that the stabilities of β-globin mRNAs carrying two different duplications of a defined 3′UTR stem-loop motif—previously identified as a determinant of mRNA stability—was significantly increased relative to the wild-type beta-globin message (by 1.5 and 2 times, respectively).

Having described preferred embodiments of the invention with reference to the accompanying drawings, it is to be understood that the invention is not limited to the precise embodiments, and that various changes and modifications may be effected therein by those skilled in the art without departing from the scope or spirit of the invention as defined in the appended claims.

What is claimed:

1. A hyperstable mRNA, comprising a stability-inducing motif at the 3′UTR of the mRNA, said stability inducing motif comprising a site specific deletion and substitution of a predetermined nucleotide sequence at the 3′UTR.

2. The hyperstable mRNA of claim 1, wherein the stability inducing motif comprises a nucleolin binding site.

3. The hyperstable mRNA of claim 1, wherein the stability inducing motif is capable of forming a stem-loop construct.

4. The hyperstable mRNA of claim 1, wherein the stability inducing motif is inserted at position 15 of the 3′UTR.

5. The hyperstable mRNA of claim 1, comprising two or more stability inducing motiffs.

6. The hyperstable mRNA of claim 3 or 5, wherein the nucleolin binding site is inserted at the right half-stem of a stem-loop construct comprising the stability-inducing motif.

7. The hyperstable mRNA of claim 1 or 5, wherein the stability inducing motif is comprised of between about 55 and 80 nucleotides.

8. The hyperstable mRNA of claim 1 or 5, wherein the stability inducing motif comprises the sequence set forth in SEQ ID NO: 1.

9. The hyperstable mRNA of claim 1 or 5, wherein the stability inducing motif comprises the sequence set forth in SEQ ID NO: 2.

10. The hyperstable mRNA of claim 1 or 5, wherein the stability inducing motif comprises the sequence set forth in SEQ ID NO: 3.

11. The hyperstable mRNA of claim 1 or 5, wherein the stability inducing motif comprises the sequence set forth in SEQ ID No.'s 1 and No. 2

12. The hyperstable mRNA of claim 1, wherein the mRNA is a β-globin mRNA

13. The hyperstable mRNA of claim 12, wherein the native DNA sequence comprises a deletion of the sequence set forth in SEQ ID No.5

14. The hyperstable mRNA of claim 13, wherein the native DNA sequence comprises an insertion of the sequence set forth in SEQ ID No.6 at position 561 of the native DNA.

15. A method of increasing the stability of a mRNA molecule, comprising the step of inserting a stability inducing motif at the 3′UTR of said mRNA molecule, thereby increasing the stability of a mRNA molecule.

16. The method of claim 15, wherein said stability inducing motif comprises SEQ ID NO: 1, SEQ ID NO: 2, SEQ ID NO: 3, or a combination thereof.

17. The method of claim 15, wherein said stability inducing motif comprises a nucleolin binding site.

18. The method of claim 17, wherein said nucleolin binding site is a nucleolin beta-globin binding site.

19. The method of claim 15, whereby the stability inducing motif is a stem-loop construct comprising SEQ ID NO. 1, 2, 3 or their combination, is inserted at the ′3UTR of the mRNA molecule, at a predetermined location on the 5′ side of the wild-type existing stability inducing motif.

20. A method of increasing the amount of a mRNA molecule in a cell, comprising the step of inserting a stability inducing motif at the 3′UTR of said mRNA molecule, thereby increasing the amount of a mRNA molecule in a cell.

21. The method of claim 20, further comprising the step of increasing the expression rate of said mRNA molecule.

22. The method of claim 21, whereby said increasing the expression rate of said mRNA molecule comprises manipulating a gene promoter element.

23. The method of claim 21, whereby increasing the amount of said mRNA molecule in said cell comprises larger production of protein translated from said mRNA molecule in said cell.

24. The method of claim 21, whereby said stability inducing motif is a beta globin stability inducing motif.

25. The method of claim 21, whereby said stability inducing motif comprises SEQ ID NO: 1, SEQ ID NO: 2, SEQ ID NO: 3, or a combination thereof.

26. The method of claim 15, whereby the stability inducing motif is a stem-loop construct comprising SEQ ID NO. 1, 2, 3 or their combination, is inserted at the ′3UTR of the mRNA molecule, at a predetermined location on the 5′ side of the wild-type existing stability inducing motif.

27. The method of claim 20, further comprising inserting an additional stability inducing motif, whereby the additional stability inducing motif comprises the sequence set forth in SEQ ID No.s 1, 2, or 3 or a combination thereof.

28. A method of producing an exogenous protein in a eukaryotic cell, comprising the step of inserting a stability inducing motif at the 3′UTR of a mRNA molecule encoding said protein, thereby producing an exogenous protein in a eukaryotic cell.

29. The method of claim 28, further comprising the step of increasing the expression rate of said mRNA molecule.

30. The method of claim 29, whereby said increasing the expression rate of said mRNA molecule comprises manipulating a gene promoter element.

31. The method of claim 29, whereby said stability inducing motif is a beta globin stability inducing motif.

32. The method of claim 29, whereby said stability inducing motif comprises SEQ ID NO: 1, SEQ ID NO: 2, SEQ ID NO: 3, or a combination thereof.

33. The method of claim 29, whereby said stability inducing motif comprises a nucleolin binding site.

34. The method of claim 29, whereby inserting a stability inducing motif at the 3′UTR of said mRNA molecule comprises increasing the stability of said mRNA.

35. The method of claim 28, whereby the stability inducing motif is a stem-loop construct comprising SEQ ID NO. 1, 2, 3 or their combination, is inserted at the ′3UTR of the mRNA molecule, at a predetermined location on the 5′ side of the wild-type existing stability inducing motif.

36. The method of claim 28, further comprising inserting an additional stability inducing motif, whereby the additional stability inducing motif comprises the sequence set forth in SEQ ID No.s 1, 2, or 3 or a combination thereof.

37. A method of treating thalassemia in a subject comprising the step of administering to the subject a DNA construct encoding a hyperstabilized beta-globin mRNA, whereby the hyperstabilized beta-globin mRNA comprises a site specific deletion and substitution of a predetermined nucleotide sequence at the 3′UTR.

38. The method of claim 37, whereby the DNA construct encodes one or more additional stability inducing motifs.

39. The method of claim 37 or 38, whereby said stability inducing motif comprises SEQ ID NO: 1, SEQ ID NO: 2, SEQ ID NO: 3, or a combination thereof.

40. The method of claim 37 or 38, whereby said stability inducing motif comprises a nucleolin binding site.

41. The method of claim 34, whereby said nucleolin binding site is a nucleolin beta-globin binding site.

42. The method of claim 37 or 38, whereby the stability inducing motif is a stem-loop construct comprising SEQ ID NO.'s 1, 2, 3 or their combination, is inserted at the ′3UTR of the mRNA molecule, at a predetermined location on the 5′ side of the wild-type existing stability inducing motif.

43. The method of claim 37, whereby the thalassemia is the result of a beta-globin mutated mRNA.

44. The method of claim 43, further comprising administering to the subject an agent capable of inhibiting the expression of the mutated beta-globin mRNA or its encoded protein.

45. The method of claim 37 or 38, whereby the native β-globin DNA sequence comprises a deletion of the sequence set forth in SEQ ID No.5

46. The method of claim 45, whereby the native β-globin DNA sequence comprises an insertion of the sequence set forth in SEQ ID No.6 at position 561 of the native DNA.

47. The method of claim 37 or 38, whereby the stability inducing motif comprises the sequence set forth in SEQ ID NO: 1.

48. The method of claim 37 or 38, whereby the stability inducing motif comprises the sequence set forth in SEQ ID NO: 2.

49. The method of claim 37 or 38, whereby the stability inducing motif comprises the sequence set forth in SEQ ID NO: 1 and the sequence set forth in SEQ ID NO: 2.

50. The method of claim 37 or 38, whereby the stability inducing motif comprises the sequence set forth in SEQ ID NO: 3.

51. A method of treating hemoglobinopathy associated with β-globin comprising the step of administering to the subject a DNA construct encoding a hyperstabilized beta-globin mRNA, whereby the hyperstabilized beta-globin mRNA comprises a site specific deletion and substitution of a predetermined nucleotide sequence at the 3′UTR.

52. The method of claim 51, whereby the DNA construct encodes one or more additional stability inducing motifs

53. The method of claim 51 or 52, whereby said stability inducing motif comprises SEQ ID NO: 1, SEQ ID NO: 2, SEQ ID NO: 3, or a combination thereof.

54. The method of claim 51 or 52, whereby said stability inducing motif comprises a nucleolin binding site.

55. The method of claim 54, whereby said nucleolin binding site is a nucleolin beta-globin binding site.

56. The method of claim 51 or 52, whereby the stability inducing motif is a stem-loop construct comprising SEQ ID NO. 1, 2, 3 or their combination, is inserted at the ′3UTR of the mRNA molecule, at a predetermined location on the 5′ side of the wild-type existing stability inducing motif.

57. The method of claim 51, whereby the thalassemia is the result of a beta-globin mutated mRNA.

58. The method of claim 57, further comprising administering to the subject an agent capable of inhibiting the expression of a mutated beta-globin mRNA or its encoded protein.

59. The method of claim 51 or 52, whereby the native β-globin DNA sequence comprises a deletion of the sequence set forth in SEQ ID No.5

60. The method of claim 59, whereby the native β-globin DNA sequence comprises an insertion of the sequence set forth in SEQ ID No.6 at position 561 of the native DNA.

61. The method of claim 51 or 52, whereby the stability inducing motif comprises the sequence set forth in SEQ ID NO: 1.

62. The method of claim 51 or 52, whereby the stability inducing motif comprises the sequence set forth in SEQ ID NO: 2.

63. The method of claim 51 or 52, whereby the stability inducing motif comprises the sequence set forth in SEQ ID NO: 1 and the sequence set forth in SEQ ID NO: 2.

64. The method of claim 51 or 52, whereby the stability inducing motif comprises the sequence set forth in SEQ ID NO: 3.

65. A method of increasing translational efficiency of mRNA in a cell, comprising the step of inserting a stability inducing motif at the 3′UTR of said mRNA molecule, wherein said stability inducing motif comprising a site specific deletion and substitution of a predetermined nucleotide sequence at the 3′UTR.

66. The method of claim 65, further comprising inserting one or more additional stability inducing motif into the 3′UTR

67. The method of claim 65 or 66, whereby said stability inducing motif comprises SEQ ID NO: 1, SEQ ID NO: 2, SEQ ID NO: 3, or a combination thereof.

68. The method of claim 65 or 66, whereby said stability inducing motif comprises a nucleolin binding site.

69. The method of claim 68, whereby said nucleolin binding site is a nucleolin beta-globin binding site.

70. The method of claim 65 or 66, whereby the stability inducing motif comprises the sequence set forth in SEQ ID NO: 1.

71. The method of claim 65 or 66, whereby the stability inducing motif comprises the sequence set forth in SEQ ID NO: 2.

72. The method of claim 65 or 66, whereby the stability inducing motif comprises the sequence set forth in SEQ ID NO: 1 and the sequence set forth in SEQ ID NO: 2.

73. The method of claim 65 or 66, whereby the stability inducing motif comprises the sequence set forth in SEQ ID NO: 3.

74. The method of claim 65 or 66, whereby the stability inducing motif comprises a cis-acting Pyrimidine-rich element (PRE)

75. The method of claim 74, whereby the native β-globin DNA sequence comprises a deletion of the sequence set forth in SEQ ID No.5

76. The method of claim 80, whereby the native β-globin DNA sequence comprises an insertion of the sequence set forth in SEQ ID No.6 at position 561 of the native DNA.
